SEQUENCE LISTING (55 sequences)

SEQ ID NO: 1 - DNA, 4680 bp, Homo sapiens
cagataccccaggctgaaggaagggccaagccctggggggaagatgggtgggcgggacaggccaagggctctgggccaagatggagctgccctcaagcctgcacacctgccctggggccgaggaaacacatgcccagtggccacgctgtagggctcagggtgggtgggacgtcaccagagctgcctggaagaaggaagttgttgaaaggtcaaaccggaatggcccgagggaaggccgcgcaggccagggccccagatggttcctgtcagggaagtggcgggcgcagctgcaggcctccggccccggcattatcacggggacacagctggctgcctcacccgcaggctgcagggagaccttccccagcctgcagccccaggcccgccccgcgtcacatgagccccagggctcccaccccctccccagggcagaggacacccagttggtggccgggagggcctcggctttccagggacagaggcccaactccaggacgccccagctggcccagcccctcctctttccctcaaggctgcaggaggtcgggaaaggcagtcctggtagaggcctgtcctgggctccaggttggcccctgagggtggccctcctcatgccggcttcaagactgagggacagggcagccagttcagcctcgggatccacctgtggctccatgtcccagacgcaccctgtgctggagagcggcctcctggcatctgccggctgctccgcaccccggggtcccaggaagggcggcccagccccagtggacaggaaggctaaggcctcagcgatgccggactccccagcggaggtgaagacgcagccccggtccacaccccccagcatgccgcccccaccgcctgccgcatcccagggggccacacgccccccctccttcacgccacacacacatcgagaggacgggcctgcgacgctgccccacggccgttttcatggctgcttaaaatggtctatggtctgtctcttgatgaacggcagcagccactcaccaacagccatcaatggtgcaccgtgcacacccaacggcttcagcaatggcccggccacctcgtccacagcctccttgtccacacagcacctgcccccagcctgcggggcccggcagctcagcaagctcaagcgcttcctcaccacactgcagcagtttggcagcgacatctccccagagattggggagcgcgtgcgcacactggtgctgggcctggtgaactcgacattgacgatcgaggagtttcattccaagcttcaggaggccaccaacttccctctgcggccgtttgtcattcccttcctgaaggcaaacctgcccttgctgcagcgggagctcctgcactgtgcacgcctggccaagcagacgcccgcccagtacttggcccagcatgagcagctcctgctggacgccagcgcctcctcccccatcgactcctcagagctgctactggaagtcaacgagaacggcaagaggaggacgcccgacaggaccaaagagaacgggtcagaccgcgacccgctgcaccccgagcacctcagcaaacggccatgcaccctgaaccctgcccagcgctacagccccagcaacgggccaccgcagcccacaccgccgccgcactaccgcctggaggacatagccatggcccaccacttccgagatgcctaccgccacccagacccccgggagctacgagagcgccatcggccgcttgtggtgcctgggtcccggcaggaagaagtgatcgaccacaagctcacagagcgtgagtgggcagaagagtggaagcacctcaacaacctcctgaactgcatcatggacatggtggagaagacgcggcgctcgctcacggtgctgcgcaggtgccaggaggccgaccgcggggagctcaaccactgggcgcggcgctacagcgacgccgaggacacaaagaagggccccgctcccgccgcggcccggccccgcagcagctccgccggtcccgaagggcctcagctagacgtgcctcgcgagttcctgccgaggaccctcaccggctacgtgcctgaggacatctggaggaaggctgaagaggccgtgaatgaggtgaagcggcaggccatgtcggagctgcagaaagccgtgtcggacgcggagcgcaaagcgcacgagctcatcaccacggagcgtgccaagatggagcgggccctggccgaggcgaagcggcaggcctccgaggacgccctgacggtcatcaaccagcaggaggactccagcgagagctgctggaactgcgggcggaaagccagtgagacgtgcagcggctgcaacgcggcacgctactgcgggtccttctgccagcatcgggactgggagaagcatcaccacgtgtgtggccagagcctgcagggccccacagccgtggtggccgacccggtgcctggaccgcccgaagccgcccacagcctgggcccctccctgcctgtgggtgctgccagccccagcgaagccggctctgcggggccttctcgccccggctcccccagcccacctggcccactggacaccgtgccccgctgaccccactggcccctggcctgccggacacagcaccgtgccaaccccacccagctccaggcccaccggatgctgtgcctggcctccgatgcctggcctgccagacactgcgccccgcctgacctgtgggagccgaccaattagtcactgctgctactgcccctctccgaaagaagacacagaaccaacaaaaccgcattcagtgcacctgcctcagctacctaatgattccgcgcggagacctcctgacaacgtctcttcaagcatcctcagaagcctcgactgagctttagacagcagagcagatgccgcaggcgcggcggctctgcccacctctcttttcctctctgtctgtctctccccctctgtcttctctatcctctctctctctatgactatcacacactttctcttcaatgaaaaaatcgaattggtggcttatattttcagcaaagaattttggggggttttgtgtgttggcaaaagagctactcagaaatggacaaagaaaacgggggggttctccccctcctgattaaaaagggagaaagaaaactgcgattttatagctggagatctgaacccagctgtgcccctcccccaggggcgtgaggctgatcagcgaagacgggaggaaagatttcgatttctgactcaagatgcatttttggtttcagatttttttttcctgtaatgttaaactctttggctttaagtaaaaatccaaaaagtttttttaaaaaagcaaaggaagcatacttgtgaactaccttgctagctagccagccaaggataccggacacacctctgctccaaaggaaatccaaaaaagcaaacacaagaaatcaaaatccaaaatttgtttgtcactgccaaagtatttttttcactgtttcacttgctcttgggtttgtttggatgtgggtctttttctcttctgttctgattttgtttgtgggtgtcgggatatttgggtgcagagggtttgtgcccagttagaagcgacttttgttctcttctgcgtaggcgttggtgcgtccgccgcgtgtgcgtggtccgtgtgccgttgctccggcctgcgtctccatatgtgtaggaaaggacacgccgtctgtcctcacgccccctgtgacttttcatatttccgttttccacttgtggaaaaaaagtgctaaagttttcttcccagagagagcataattccgaaacaaaactgtgacaatcttttgggttgattctcgactgcttttcgagcatgcggagccagcaggcctccctgaaacactgcttctcggccagcccgtcctcctctacctctctcctctccgcgccctccgacctctctcggccccctcaccccagctccgacctctctcagccccatcgccccaactccaacctctcggccccatcgccccaccgcagctactcccctttcttccaaacttttgcagaaaaaacaaaaaaactacaaacaaaagcagccctctgcctcctccccagggaagaccctgaccgtgtacatagccctggtgctcctgcccagccacccctcagatgcgttcgcctctggccctggggtgtgtctcggtgacgttttctatcagacgtgctccctcccatcctccagccctgcccaccctccctccactcctctcaactgcctcagcgatttcaagaaggaaataaagggataaagaaattcatgcttgcaccgagtacaaggacagacagcaggcacggcccgcagcctggcatctgtgcgtgtggcgtggcccgtggcttggcatctgtgtgcgtggtgtggcccgtggcctggcatctgtgtgcgtggcgtggcccgtggcctggcatctgtgtgtgtggcgtggcccgtggcctggcatctgtgtgcgtggcgtggcccgtggcctggcatctgtgtgcgtggctatcaggagttctaggaactcagtgcaatacgggagtgacccagctactgaaccagccacgaacagcccgccagaggcctgaagctgagcgtgtacgttaatgtgaatgtatatagtctttgcagaggtccaaatgatattcatgatggtaataaacgagatgtttgccaaataaaaaa

SEQ ID NO: 2 - DNA, 4170 bp, Homo sapiens
gactctcgggcgagcgcgcggcgttggagccacaggcgcggcggctggacccggcgcgggccgcggaggccggagaccgccccgggcggggtgcgcgctttgttcccgcgcggggtggccggagccgagtccccggcatggcccaggcggccgccccgcgcgccccagccccgccgcgcgcctgagcccggtgcggcgccagaagacagcgcgcagccgcccctgagtcgtggaggcggggaccaagctggaaggagcagcgactcccggaccgagtcgcaagtgtgcgccgtccgccgcccgccggatcccccggaccccccgaccccccagtggacaggaaggctaaggcctcagcgatgccggactccccagcggaggtgaagacgcagccccggtccacaccccccagcatgccgcccccaccgcctgccgcatcccagggggccacacgccccccctccttcacgccacacacactgatgaacggcagcagccactcaccaacagccatcaatggtgcaccgtgcacacccaacggcttcagcaatggcccggccacctcgtccacagcctccttgtccacacagcacctgcccccagcctgcggggcccggcagctcagcaagctcaagcgcttcctcaccacactgcagcagtttggcagcgacatctccccagagattggggagcgcgtgcgcacactggtgctgggcctggtgaactcgacattgacgatcgaggagtttcattccaagcttcaggaggccaccaacttccctctgcggccgtttgtcattcccttcctgaaggcaaacctgcccttgctgcagcgggagctcctgcactgtgcacgcctggccaagcagacgcccgcccagtacttggcccagcatgagcagctcctgctggacgccagcgcctcctcccccatcgactcctcagagctgctactggaagtcaacgagaacggcaagaggaggacgcccgacaggaccaaagagaacgggtcagaccgcgacccgctgcaccccgagcacctcagcaaacggccatgcaccctgaaccctgcccagcgctacagccccagcaacgggccaccgcagcccacaccgccgccgcactaccgcctggaggacatagccatggcccaccacttccgagatgcctaccgccacccagacccccgggagctacgagagcgccatcggccgcttgtggtgcctgggtcccggcaggaagaagtgatcgaccacaagctcacagagcgtgagtgggcagaagagtggaagcacctcaacaacctcctgaactgcatcatggacatggtggagaagacgcggcgctcgctcacggtgctgcgcaggtgccaggaggccgaccgcggggagctcaaccactgggcgcggcgctacagcgacgccgaggacacaaagaagggccccgctcccgccgcggcccggccccgcagcagctccgccggtcccgaagggcctcagctagacgtgcctcgcgagttcctgccgaggaccctcaccggctacgtgcctgaggacatctggaggaaggctgaagaggccgtgaatgaggtgaagcggcaggccatgtcggagctgcagaaagccgtgtcggacgcggagcgcaaagcgcacgagctcatcaccacggagcgtgccaagatggagcgggccctggccgaggcgaagcggcaggcctccgaggacgccctgacggtcatcaaccagcaggaggactccagcgagagctgctggaactgcgggcggaaagccagtgagacgtgcagcggctgcaacgcggcacgctactgcgggtccttctgccagcatcgggactgggagaagcatcaccacgtgtgtggccagagcctgcagggccccacagccgtggtggccgacccggtgcctggaccgcccgaagccgcccacagcctgggcccctccctgcctgtgggtgctgccagccccagcgaagccggctctgcggggccttctcgccccggctcccccagcccacctggcccactggacaccgtgccccgctgaccccactggcccctggcctgccggacacagcaccgtgccaaccccacccagctccaggcccaccggatgctgtgcctggcctccgatgcctggcctgccagacactgcgccccgcctgacctgtgggagccgaccaattagtcactgctgctactgcccctctccgaaagaagacacagaaccaacaaaaccgcattcagtgcacctgcctcagctacctaatgattccgcgcggagacctcctgacaacgtctcttcaagcatcctcagaagcctcgactgagctttagacagcagagcagatgccgcaggcgcggcggctctgcccacctctcttttcctctctgtctgtctctccccctctgtcttctctatcctctctctctctatgactatcacacactttctcttcaatgaaaaaatcgaattggtggcttatattttcagcaaagaattttggggggttttgtgtgttggcaaaagagctactcagaaatggacaaagaaaacgggggggttctccccctcctgattaaaaagggagaaagaaaactgcgattttatagctggagatctgaacccagctgtgcccctcccccaggggcgtgaggctgatcagcgaagacgggaggaaagatttcgatttctgactcaagatgcatttttggtttcagatttttttttcctgtaatgttaaactctttggctttaagtaaaaatccaaaaagtttttttaaaaaagcaaaggaagcatacttgtgaactaccttgctagctagccagccaaggataccggacacacctctgctccaaaggaaatccaaaaaagcaaacacaagaaatcaaaatccaaaatttgtttgtcactgccaaagtatttttttcactgtttcacttgctcttgggtttgtttggatgtgggtctttttctcttctgttctgattttgtttgtgggtgtcgggatatttgggtgcagagggtttgtgcccagttagaagcgacttttgttctcttctgcgtaggcgttggtgcgtccgccgcgtgtgcgtggtccgtgtgccgttgctccggcctgcgtctccatatgtgtaggaaaggacacgccgtctgtcctcacgccccctgtgacttttcatatttccgttttccacttgtggaaaaaaagtgctaaagttttcttcccagagagagcataattccgaaacaaaactgtgacaatcttttgggttgattctcgactgcttttcgagcatgcggagccagcaggcctccctgaaacactgcttctcggccagcccgtcctcctctacctctctcctctccgcgccctccgacctctctcggccccctcaccccagctccgacctctctcagccccatcgccccaactccaacctctcggccccatcgccccaccgcagctactcccctttcttccaaacttttgcagaaaaaacaaaaaaactacaaacaaaagcagccctctgcctcctccccagggaagaccctgaccgtgtacatagccctggtgctcctgcccagccacccctcagatgcgttcgcctctggccctggggtgtgtctcggtgacgttttctatcagacgtgctccctcccatcctccagccctgcccaccctccctccactcctctcaactgcctcagcgatttcaagaaggaaataaagggataaagaaattcatgcttgcaccgagtacaaggacagacagcaggcacggcccgcagcctggcatctgtgcgtgtggcgtggcccgtggcttggcatctgtgtgcgtggtgtggcccgtggcctggcatctgtgtgcgtggcgtggcccgtggcctggcatctgtgtgtgtggcgtggcccgtggcctggcatctgtgtgcgtggcgtggcccgtggcctggcatctgtgtgcgtggctatcaggagttctaggaactcagtgcaatacgggagtgacccagctactgaaccagccacgaacagcccgccagaggcctgaagctgagcgtgtacgttaatgtgaatgtatatagtctttgcagaggtccaaatgatattcatgatggtaataaacgagatgtttgccaaataaaaaa

SEQ ID NO: 3 - protein, 830 aa, Homo sapiens
MELPSSLHTCPGAEETHAQWPRCRAQGGWDVTRAAWKKEVVERSNRNGPREGRAGQGPRWFLSGKWRAQLQASGPGIITGTQLAASPAGCRETFPSLQPQARPASHEPQGSHPLPRAEDTQLVAGRASAFQGQRPNSRTPQLAQPLLFPSRLQEVGKGSPGRGLSWAPGWPLRVALLMPASRLRDRAASSASGSTCGSMSQTHPVLESGLLASAGCSAPRGPRKGGPAPVDRKAKASAMPDSPAEVKTQPRSTPPSMPPPPPAASQGATRPPSFTPHTHREDGPATLPHGRFHGCLKWSMVCLLMNGSSHSPTAINGAPCTPNGFSNGPATSSTASLSTQHLPPACGARQLSKLKRFLTTLQQFGSDISPEIGERVRTLVLGLVNSTLTIEEFHSKLQEATNFPLRPFVIPFLKANLPLLQRELLHCARLAKQTPAQYLAQHEQLLLDASASSPIDSSELLLEVNENGKRRTPDRTKENGSDRDPLHPEHLSKRPCTLNPAQRYSPSNGPPQPTPPPHYRLEDIAMAHHFRDAYRHPDPRELRERHRPLVVPGSRQEEVIDHKLTEREWAEEWKHLNNLLNCIMDMVEKTRRSLTVLRRCQEADRGELNHWARRYSDAEDTKKGPAPAAARPRSSSAGPEGPQLDVPREFLPRTLTGYVPEDIWRKAEEAVNEVKRQAMSELQKAVSDAERKAHELITTERAKMERALAEAKRQASEDALTVINQQEDSSESCWNCGRKASETCSGCNAARYCGSFCQHRDWEKHHHVCGQSLQGPTAVVADPVPGPPEAAHSLGPSLPVGAASPSEAGSAGPSRPGSPSPPGPLDTVPR

SEQ ID NO: 4 - protein, 567 aa, Homo sapiens
MPDSPAEVKTQPRSTPPSMPPPPPAASQGATRPPSFTPHTLMNGSSHSPTAINGAPCTPNGFSNGPATSSTASLSTQHLPPACGARQLSKLKRFLTTLQQFGSDISPEIGERVRTLVLGLVNSTLTIEEFHSKLQEATNFPLRPFVIPFLKANLPLLQRELLHCARLAKQTPAQYLAQHEQLLLDASASSPIDSSELLLEVNENGKRRTPDRTKENGSDRDPLHPEHLSKRPCTLNPAQRYSPSNGPPQPTPPPHYRLEDIAMAHHFRDAYRHPDPRELRERHRPLVVPGSRQEEVIDHKLTEREWAEEWKHLNNLLNCIMDMVEKTRRSLTVLRRCQEADRGELNHWARRYSDAEDTKKGPAPAAARPRSSSAGPEGPQLDVPREFLPRTLTGYVPEDIWRKAEEAVNEVKRQAMSELQKAVSDAERKAHELITTERAKMERALAEAKRQASEDALTVINQQEDSSESCWNCGRKASETCSGCNAARYCGSFCQHRDWEKHHHVCGQSLQGPTAVVADPVPGPPEAAHSLGPSLPVGAASPSEAGSAGPSRPGSPSPPGPLDTVPR

SEQ ID NO: 5 - DNA, 20 bp, Homo sapiens
gggtttgtgcccagttagaa

SEQ ID NO: 6 - DNA, 20 bp, Homo sapiens
tatgaaaagtcacagggggc

SEQ ID NO: 7 - DNA, 25 bp, Homo sapiens
ggagcttccccaactcataaatgcc

SEQ ID NO: 8 - DNA, 25 bp, Homo sapiens
gcatgatgtctgatgtggtcagtaa

SEQ ID NO: 9 - DNA, 22 bp, Homo sapiens
ggcaaatgctggacccaacaaa

SEQ ID NO: 10 - DNA, 25 bp, Homo sapiens
ctaggcatgggagggaacaagggaa

SEQ ID NO: 11 - DNA, 21 bp, Homo sapiens
gactgggctgcgtgctcatcc

SEQ ID NO: 12 - DNA, 24 bp, Homo sapiens
aggccctgtggtcactcatactgc

SEQ ID NO: 13 - DNA, 20 bp, Homo sapiens
aggggctaacaatggacacc

SEQ ID NO: 14 - DNA, 22 bp, Homo sapiens
ccgaagataagggggaactact

SEQ ID NO: 15 - DNA, 24 bp, Homo sapiens
gggcctggtgaactcgacattgac

SEQ ID NO: 16 - DNA, 22 bp, Homo sapiens
acggccgcagagggaagttggt

SEQ ID NO: 17 - DNA, 18 bp, Homo sapiens
gtcctgggctccaggttg

SEQ ID NO: 18 - DNA, 20 bp, Homo sapiens
gaagctctaaggagtcacag

SEQ ID NO: 19 - DNA, 20 bp, Homo sapiens
ttgcacttagcctgcttcac

SEQ ID NO: 20 - DNA, 19 bp, Homo sapiens
gcctcccctgaaacacctg

SEQ ID NO: 21 - DNA, 22 bp, Homo sapiens
aaaaatcactctgagaagtagg

SEQ ID NO: 22 - DNA, 20 bp, Homo sapiens
tgttgggccagctgaggatg

SEQ ID NO: 23 - DNA, 20 bp, Homo sapiens
tgtgtcctcatgtccgcttc

SEQ ID NO: 24 - DNA, 20 bp, Homo sapiens
cggagggaatatgcatgtcc

SEQ ID NO: 25 - DNA, 19 bp, Homo sapiens
ctgcctccaacacggaagc

SEQ ID NO: 26 - DNA, 19 bp, Homo sapiens
tccacgctgcgaaggagtg

SEQ ID NO: 27 - DNA, 19 bp, Homo sapiens
gtgcacccctgcatgctac

SEQ ID NO: 28 - DNA, 20 bp, Homo sapiens
gaggaggttccctctcttac

SEQ ID NO: 29 - DNA, 20 bp, Homo sapiens
gttcatcctatgtccactgc

SEQ ID NO: 30 - DNA, 20 bp, Homo sapiens
catgtgtgctcctgtaacac

SEQ ID NO: 31 - DNA, 20 bp, Homo sapiens
agaatagggcagagactggc

SEQ ID NO: 32 - DNA, 19 bp, Homo sapiens
tggctgtgtgtggacactc

SEQ ID NO: 33 - DNA, 19 bp, Homo sapiens
tctgaggtgctgaaggctg

SEQ ID NO: 34 - DNA, 18 bp, Homo sapiens
agcaccccgtgtctgctc

SEQ ID NO: 35 - DNA, 19 bp, Homo sapiens
gtggcccatcctgtgtgac

SEQ ID NO: 36 - DNA, 20 bp, Homo sapiens
ttcaaagctgagccggtgag

SEQ ID NO: 37 - DNA, 19 bp, Homo sapiens
tggccacgcgtaggaagtc

SEQ ID NO: 38 - DNA, 18 bp, Homo sapiens
gcaggggatgggtgtcag

SEQ ID NO: 39 - DNA, 18 bp, Homo sapiens
cagacccagccctgactg

SEQ ID NO: 40 - DNA, 20 bp, Homo sapiens
cacacgtggtgatgcttctc

SEQ ID NO: 41 - DNA, 19 bp, Homo sapiens
tctgccagcatcgggactg

SEQ ID NO: 42 - DNA, 19 bp, Homo sapiens
gttggcacggtgctgtgtc

SEQ ID NO: 43 - DNA, 19 bp, Homo sapiens
gacagcagagcagatgccg

SEQ ID NO: 44 - DNA, 21 bp, Homo sapiens
gcaaggtagttcacaagtatg

SEQ ID NO: 45 - DNA, 21 bp, Homo sapiens
ggcggcaccaccatgtaccct

SEQ ID NO: 46 - DNA, 21 bp, Homo sapiens
aggggccggactcgtcatact

SEQ ID NO: 47 - DNA, 53 bp, Homo sapiens
atggagcagaagctgatcagcgaggaggacctgatgccggactccccagcgga

SEQ ID NO: 48 - DNA, 20 bp, Homo sapiens
tcagcggggcacggtgtcca

SEQ ID NO: 49 - DNA, 20 bp, Homo sapiens
atgccggactccccagcgga

SEQ ID NO: 50 - DNA, 28 bp, Homo sapiens
gcggggtgcgcgctttgttcccgcgcgg

SEQ ID NO: 51 - DNA, 28 bp, Homo sapiens
cgagactcccagcgcccgggcgcgtcgt

SEQ ID NO: 52 - DNA, 28 bp, Homo sapiens
gcggggtcggcgttttgttttcgcgcgg

SEQ ID NO: 53 - DNA, 28 bp, Homo sapiens
cgaaactcccaacgcccgaacgcgtcgt

SEQ ID NO: 54 - DNA, 22 bp, Homo sapiens
tgtgtgttttgtttttgtgtgg

SEQ ID NO: 55 - DNA, 24 bp, Homo sapiens
actcccaacacccaaacacatcat

(19) United States
(12) Patent Application Publication
(10) Pub. No.: US 2005/0107313 A1
(43) Pub. Date: May 19, 2005

(54) Tumour suppressor gene

(75) Inventors: David Frederick Callen, Malvern (AU); Scott Anthony Whitmore, Golden Grove (AU); Gabriel Kremmidiotis, Aberfoyle Park (AU); Marina Kochetkova, Medindie (AU); Joanna Crawford, Stirling (AU)

Correspondence Address: JENKINS, WILSON & TAYLOR, P. A., 3100 TOWER BLVD, SUITE 1400, DURHAM, NC, 27707, US

(73) Assignee: Bionomics Limited, Thebarton, SA, (AU)

(21) Appl. No.: 10/362,820

(22) PCT Filed: Aug. 31, 2001
(86) PCT No.: PCT/AU01/01097

(30) Foreign Application Priority Data
Sep. 1, 2000 (AU) PQ 9806

(51) Int. Cl. C12Q 1/68; C07H 21/04; A61K 48/00; C12N 9/99

(52) U.S. Cl. 514/44; 536/23.2; 435/6; 435/69.1; 435/320.1; 435/325; 435/184

(57) ABSTRACT

The invention provides an isolated DNA molecule comprising the nucleotide sequence set forth in SEQ ID NO: 1 or 2, or active fragments thereof, which encode a polypeptide active in suppressing cellular functions associated with cancer. In particular, the polypeptide (MTG16) functions as a tumour suppressor gene. It also provides variants of such DNA molecules which retain their function, polypeptides encoded by the DNA molecules and antibodies thereto, as well as the use of these molecules in diagnostic, prognostic and therapeutic procedures and other uses such as screening for candidate pharmaceuticals and animal model generation.

### TECHNICAL FIELD

The present invention relates to a gene, MTG16, which has been mapped to the tip of the long arm of chromosome 16 at 16q24.3. A novel function of the MTG16 gene has been defined. The MTG16 gene encodes a polypeptide that has a tumour suppressor function. In view of the realisation that MTG16 has a tumour suppressor function, the invention is also concerned with the diagnosis of cancer, in particular breast, prostate, ovarian and hepatocellular carcinoma, cancer therapy and screening of drugs for anti-tumour activity.

### BACKGROUND ART

The development of human carcinomas has been shown to arise from the accumulation of genetic changes involving both positive regulators of cell function (oncogenes) and negative regulators (tumour suppressor genes). For a normal somatic cell to evolve into a metastatic tumour it requires multiple changes at the cellular level, such as immortalisation, loss of contact inhibition and invasive growth capacity, and changes at the tissue level, such as evasion of host immune responses and growth restraints imposed by surrounding cells, and the formation of a blood supply for the growing tumour.

Molecular genetic studies of colorectal carcinoma have provided substantial evidence that the generation of malignancy requires the sequential accumulation of a number of genetic changes within the epithelial stem cell of the colon. For a normal colonic epithelial cell to become a benign adenoma, progress to intermediate and late adenomas, and finally become a malignant cell, inactivating mutations in tumour suppressor genes and activating mutations in proto-oncogenes are required (Fearon and Vogelstein, 1990).

Tumour suppressor genes were first identified in the childhood cancer retinoblastoma. Both inherited and sporadic forms of this cancer exist, with the familial form inherited as a highly penetrant autosomal dominant trait, which was mapped to chromosome 13q14 by genetic linkage analysis (Sparkes et al., 1983). The observation that bilateral retinoblastoma was characteristic of the inherited disease and occurred at an early age, whereas unilateral retinoblastoma was characteristic of the sporadic form and occurred at a later age, led to the hypothesis that the tumour arises from two mutational steps (Knudson, 1971). With this proposition, familial cancers would result from an inherited germline mutation of a gene suppressing the growth of cells (tumour suppressor gene), such that all cells would carry this mutation. A second mutation or “hit” in any cell therefore resulted in the manifestation of the recessive mutation leading to cancer. The fact that only one more “hit” produces a cancerous cell meant that individuals with an inherited pre-disposition to the disease had an earlier age of onset and often bilateral tumours. In contrast, sporadic cases tended to be in one eye and later in onset because two “hits” were needed to the genes in the same cell.

This hypothesis was confirmed with the use of genetic markers mapping to 13q14 to type DNA isolated from blood and tumour samples taken from the same affected individuals (Cavenee et al., 1983). In several cases the constitutional DNA from lymphocytes was heterozygous for some markers but the tumour cells appeared homozygous for the same markers. The apparent reduction to homozygosity (or loss of heterozygosity, LOH) through the loss of one allele of these markers was suggested to be the second “hit” which was removing the remaining functional copy of the retinoblastoma gene in these individuals. The analysis of tumours in familial cases showed that the chromosome from the unaffected parent was in each instance the one eliminated from the tumour. A number of mechanisms were proposed including mitotic recombination, mitotic non-disjunction with loss of the wild-type allele or reduplication of the mutant allele, and gene conversion, deletion or mutation.

In addition to retinoblastoma, studies of other cancers have supported the model that LOH is a specific event in the pathogenesis of cancer. In Von Hippel-Lindau (VHL) syndrome both sporadic and inherited cases of the syndrome show LOH for the short arm of chromosome 3. Somatic translocations involving 3p in sporadic tumours, and genetic linkage to the same region in affected families has also been observed. Similarly, in colorectal carcinoma, inherited forms of the disease have been mapped to the long arm of chromosome 5 while LOH at 5q has been reported in both the familial and sporadic versions of the disease and the APC gene, mapping to this region, has been shown to be involved (Groden et al., 1991). Other examples, which include the TP53 and NF2 genes, firmly establish the fact that a general mechanism in human cancer is the inactivation of tumour suppressor genes by LOH. Indeed LOH in tumour DNA is now taken as being strongly indicative of the presence and inactivation of a tumour suppressor gene.

Breast cancer is the most common malignancy seen in women, affecting approximately 10% of females in the Western world. The route to breast cancer is not as well mapped as that of colon cancer due in part to the histological stages of breast cancer development being less well defined. It is known however, that breast cancer is derived from the epithelial lining of terminal mammary ducts or lobuli. Hormonal influences, such as those exerted by oestrogen, are believed to be important because of the marked increase in breast cancer incidence in post-menopausal women, but the initial steps in breast cancer development probably occur before the onset of menopause. As with colon carcinoma, it is believed that a number of genes need to become involved in a stepwise progression during breast tumourigenesis.

Certain women appear to be at an increased risk of developing breast cancer. Genetic linkage analysis has shown that 5 to 10% of all breast cancers are due to one of at least two autosomal dominant susceptibility genes. Generally, women carrying a mutation in a susceptibility gene develop breast cancer at a younger age compared to the general population, often have bilateral breast tumours, and are at an increased risk of developing cancers in other organs, particularly carcinoma of the ovary.

Genetic linkage analysis of families showing a high incidence of early-onset breast cancer (before the age of 46) was successful in mapping the first susceptibility gene, BRCA1, to chromosome 17q21 (Hall et al., 1990). Subsequent to this, the BRCA2 gene was mapped to chromosome 13q12-q13 (Wooster et al., 1994) with this gene conferring a higher incidence of male breast cancer and a lower incidence of ovarian cancer when compared to BRCA1.

Both BRCA1 and BRCA2 have since been cloned (Miki et al., 1994; Wooster et al., 1995) and numerous mutations have been identified in these genes in susceptible individuals with familial cases of breast cancer.

Additional inherited breast cancer syndromes exist, however they are rare. Inherited mutations in the TP53 gene have been identified in individuals with Li-Fraumeni syndrome, a familial cancer resulting in epithelial neoplasms occurring at multiple sites including the breast. Similarly, germline mutations in the MMAC1/PTEN gene involved in Cowden's disease and the ataxia telangiectasia (AT) gene have been shown to confer an increased risk of developing breast cancer, among other clinical manifestations, but together account for only a small percentage of families with an inherited predisposition to breast cancer.

Somatic mutations in the TP53 gene have been shown to occur in a high percentage of individuals with sporadic breast cancer. However, although LOH has been observed at the BRCA1 and BRCA2 loci at a frequency of 30 to 40% in sporadic cases (Cleton-Jansen et al., 1995; Saito et al., 1993), there is virtually no sign of somatic mutations in the retained allele of these two genes in sporadic cancers (Futreal et al., 1994; Miki et al., 1996). Recent data suggests that DNA methylation of the promoter sequences of these genes may be an important mechanism of down-regulation. The use of both restriction fragment length polymorphisms and small tandem repeat polymorphism markers has identified numerous regions of allelic imbalance in breast cancer suggesting the presence of additional tumour suppressor genes, which may be implicated in breast cancer. Data compiled from more than 30 studies reveals the loss of DNA from at least 11 chromosome arms at a frequency of more than 25%, with regions such as 16q and 17p affected in more than 50% of tumours (Devilee and Cornelisse, 1994; Brenner and Aldaz, 1995). However only some of these regions are known to harbour tumour suppressor genes shown to be mutated in individuals with both sporadic (TP53 and RB genes) and familial (TP53, RB, BRCA1, and BRCA2 genes) forms of breast cancer.

Cytogenetic studies have implicated loss of the long arm of chromosome 16 as an early event in breast carcinogenesis since it is found in tumours with few or no other cytogenetic abnormalities. Alterations in chromosome 1 and 16 have also been seen in several cases of ductal carcinoma in situ (DCIS), the preinvasive stage of ductal breast carcinoma. In addition, LOH studies on DCIS samples identified loss of 16q markers in 29 to 89% of the cases tested (Chen et al., 1996; Radford et al., 1995). Together, these findings suggest the presence of a tumour suppressor gene mapping to the long arm of chromosome 16 that is critically involved in the early development of a large proportion of breast cancers, but to date no such gene has been identified.

### DISCLOSURE OF THE INVENTION

According to one aspect of the present invention there is provided an isolated mammalian DNA molecule encoding the MTG16 gene which is a novel tumour suppressor gene.

According to another aspect of the present invention, there is provided an isolated mammalian DNA molecule encoding MTG16a or MTG16b having the nucleotide sequences set forth in SEQ ID Numbers: 1 or 2 respectively. It will be appreciated that the sequences shown in SEQ ID Numbers: 1 and 2 are novel. The MTG16a sequence (SEQ ID NO: 1) includes nucleotides encoding an additional 177 amino acids at the 5′ end of the gene when compared to the sequence originally proposed by Gamou et al., 1998. The sequence listed for MTG16b (SEQ ID NO: 2) differs from that previously disclosed by Gamou et al., 1998 in that it includes additional 5′ untranslated region sequence in which can be identified a CpG island. Abnormal methylation of the CpG island may be one mechanism for inactivation of MTG16b.

The present invention also provides an isolated mammalian DNA molecule comprising the nucleotide sequence set forth in SEQ ID NO: 1 or 2, or a fragment thereof, which encodes a polypeptide active in suppressing cellular functions associated with cancer. It will be understood that cellular functions associated with cancer include but are not restricted to, cell proliferation, cell cycle, cell survival, invasion and growth receptor responses. The suppression of these cellular functions is frequently referred to as tumour suppression function and the genes which encode proteins having this function as tumour suppressor genes.

The invention also encompasses an isolated mammalian DNA molecule that is at least 75% identical to a DNA molecule consisting of the nucleotide sequence set forth in SEQ ID NO: 1 or 2 and which encodes a polypeptide active in suppressing cellular functions associated with cancer, including but not restricted to, cell proliferation, cell cycle, cell survival, invasion and growth receptor responses.

Such variants will have preferably at least about 85%, and most preferably at least about 95% sequence identity to the nucleotide sequence encoding MTG16. A particular aspect of the invention encompasses a variant of SEQ ID NO: 1 or 2 which has at least about 75%, more preferably at least about 85%, and most preferably at least about 95% sequence identity to SEQ ID NO: 1 or 2. Any one of the polynucleotide variants described above can encode an amino acid sequence, which contains at least one functional or structural characteristic of MTG16.

Typically, sequence identity is calculated using the BLASTN algorithm with the BLOSSUM62 default matrix.

The invention also encompasses an isolated mammalian DNA molecule that encodes a polypeptide active in suppressing cellular functions associated with cancer, including but not restricted to, cell proliferation, cell cycle, cell survival, invasion and growth receptor responses, and which hybridizes under stringent conditions with a DNA molecule consisting of the nucleotide sequence set forth in SEQ ID NO: 1 or 2.

Under stringent conditions, hybridization will most preferably occur at 42° C. in 750 mM NaCl, 75 mM trisodium citrate, 2% SDS, 50% formamide, 1× Denhardt's (0.02% (w/v) Ficoll 400; 0.02% (w/v) polyvinylpirolidone; 0.02% (w/v) BSA), 10% (w/v) dextran sulphate and 100 ug/ml denatured salmon sperm DNA. Useful variations on these conditions will be readily apparent to those skilled in the art. The washing steps which follow hybridization most preferably occur at 65° C. in 15 mM NaCl, 1.5 mM trisodium citrate, and 1% SDS. Additional variations on these conditions will be readily apparent to those skilled in the art.

The invention also provides an isolated mammalian DNA molecule which encodes a polypeptide having the amino acid sequence set forth in SEQ ID NO: 3.

Still further, the invention encompasses an isolated DNA molecule wherein the amino acid sequence has at least 70%, preferably 85%, and most preferably 95%, sequence identity to the sequence set forth in SEQ ID NO: 2.

Preferably, sequence identity is determined using the BLASTP algorithm with the BLOSSUM62 default matrix.

In a further aspect the invention provides a gene, MTG16, comprising the nucleotide sequence set forth in SEQ ID NO: 1 or 2 and MTG16 control elements.

Preferably, the MTG16 control elements are those which mediate expression in breast tissue.

The nucleotide sequences of the present invention can be engineered using methods accepted in the art so as to alter MTG16-encoding sequences for a variety of purposes. These include, but are not limited to, modification of the cloning, processing, and/or expression of the gene product. PCR reassembly of gene fragments and the use of synthetic oligonucleotides allow the engineering of MTG16 nucleotide sequences. For example, oligonucleotide-mediated site-directed mutagenesis can introduce mutations that create new restriction sites, alter glycosylation patterns and produce splice variants etc.

As a result of the degeneracy of the genetic code, a number of polynucleotide sequences encoding MTG16, some that may have minimal similarity to the polynucleotide sequences of any known and naturally occurring gene, may be produced. Thus, the invention includes each and every possible variation of polynucleotide sequence that could be made by selecting combinations based on possible codon choices. These combinations are made in accordance with the standard triplet genetic code as applied to the polynucleotide sequence of naturally occurring MTG16, and all such variations are to be considered as being specifically disclosed.

The polynucleotides of this invention include RNA, cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences encoding MTG16 or its derivatives possessing a substantially different codon usage than that of the naturally occurring MTG16. For example, codons may be selected to increase the rate of expression of the peptide in a particular prokaryotic or eukaryotic host corresponding with the frequency that particular codons are utilized by the host. Other reasons to alter the nucleotide sequence encoding MTG16 and its derivatives without altering the encoded amino acid sequences include the production of RNA transcripts having more desirable properties, such as a greater half-life, than transcripts produced from the naturally occurring sequence.

The invention also encompasses production of DNA sequences, which encode MTG16 and its derivatives, or fragments thereof, entirely by synthetic chemistry. Synthetic sequences may be inserted into expression vectors and cell systems that contain the necessary elements for transcriptional and translational control of the inserted coding sequence in a suitable host. These elements may include regulatory sequences, promoters, 5′ and 3′ untranslated regions and specific initiation signals (such as an ATG initiation codon and Kozak consensus sequence) which allow more efficient translation of sequences encoding MTG16. In cases where the complete MTG16 coding sequence including its initiation codon and upstream regulatory sequences are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

Nucleic acid molecules that are complements of the sequences described herein may also be prepared.

The present invention allows for the preparation of purified MTG16 polypeptide or protein from the polynucleotides of the present invention, or variants thereof. In order to do this, host cells may be transfected with a DNA molecule as described above. Typically said host cells are transfected with an expression vector comprising a DNA molecule according to the invention. A variety of expression vector/host systems may be utilized to contain and express sequences encoding MTG16. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. Mammalian cells can also be used to express the MTG16 protein using various expression vectors including plasmid, cosmid and viral systems such as a vaccinia virus expression system. The invention is not limited by the host cell employed.

The polynucleotide sequences, or variants thereof, of the present invention can be stably expressed in cell lines to allow long term production of recombinant proteins in mammalian systems. Sequences encoding MTG16 can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode MTG16 may be designed to contain signal sequences which direct secretion of MTG16 through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of MTG16 are needed such as for antibody production, vectors which direct high levels of expression of MTG16 may be used such as those containing the T5 or T7 inducible bacteriophage promoter. The present invention also includes the use of the expression systems described above in generating and isolating fusion proteins which contain important functional domains of the protein. These fusion proteins are used for binding, structural and functional studies as well as for the generation of appropriate antibodies.

In order to express and purify the protein as a fusion protein, the appropriate MTG16 cDNA sequence is inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence and the MTG16 protein obtained by enzymatic cleavage of the fusion protein.

Fragments of MTG16 may also be produced by direct peptide synthesis using solid-phase techniques. Automated synthesis may be achieved by using the ABI 431A Peptide Synthesizer (Perkin-Elmer). Various fragments of MTG16 may be synthesized separately and then combined to produce the full length molecule.

According to the present invention there is provided an isolated mammalian polypeptide encoded by the MTG16 gene which has a novel tumour suppressor function.

According to the invention there is provided an isolated mammalian polypeptide encoded by the MTG16 gene comprising the amino acid sequence set forth in SEQ ID NO: 3.

The sequence listed corresponds to MTG16a and differs from the sequence previously disclosed by Gamou et al., 1998 as it contains an additional 177 amino acids at its 5′end.

According to a still further aspect of the invention there is provided a polypeptide, comprising the amino acid sequence set forth in SEQ ID NO: 3 or 4, or a fragment thereof, active in suppressing cellular functions associated with cancer, including but not restricted to, cell proliferation, cell cycle, cell survival, invasion and growth receptor responses.

The invention also encompasses an isolated mammalian polypeptide active in suppressing cellular functions associated with cancer, including but not restricted to, cell proliferation, cell cycle, cell survival, invasion and growth receptor responses and having at least 75%, more preferably at least 85% and most preferably at least 95% sequence identity with the amino acid sequence set forth in SEQ ID NO: 3.

Preferably, sequence identity is determined using the BLASTP algorithm with the BLOSSUM62 default matrix.

In a further aspect of the invention there is provided a method of preparing a polypeptide as described above, comprising the steps of:
(1) culturing the host cells under conditions effective for production of the polypeptide; and
(2) harvesting the polypeptide.

Substantially purified MTG16 protein or fragments thereof can then be used in further biochemical analyses to establish secondary and tertiary structure for example by x-ray crystallography of MTG16 protein or by NMR. Determination of structure allows for the rational design of pharmaceuticals to mimic or interact with the protein, alter protein charge configuration or charge interaction with other proteins, or to alter its function in the cell.

The invention has shown that the MTG16 gene is located in a region of restricted LOH observed in breast and prostate cancer. The invention has found that the expression of MTG16 is grossly reduced in a number of breast cancer cell lines and primary tumours concomitant with 16q LOH. In addition, a proline to threonine amino acid change in the coding region of MTG16 (P255T in MTG16a or P17T in MTG16b) has been detected in a breast cancer cell line. The invention has also shown that introduction of MTG16 into different breast tumour derived cell lines dramatically reduces cell growth on a plastic surface and in soft agar. The invention has localised MTG16 to cell nuclei and it has been shown that MTG16 is able to repress transcription in CAT reporter assays. As LOH of chromosome 16q has also been observed in other malignancies such as prostate, hepatocellular, ovarian and primitive neuroectodermal tumours and MTG16 is expressed in many tissues suggests that MTG16 may be a multi-tissue tumour suppressor gene.

The invention therefore enables therapeutic methods for the treatment of all diseases associated with MTG16 tumour suppressor gene function and also enables methods for the diagnosis of all diseases associated with MTG16 tumour suppressor gene function.

Examples of such diseases include, but are not limited to, cancers such as adenocarcinoma, leukaemia, lymphoma, melanoma, myeloma, sarcoma, teratocarcinoma, and, in particular, cancer of the breast, prostate, liver, ovary, neuroectoderm, placenta, skeletal muscle, tonsil, lymph tissue, kidney and colon. Other cancers may include those of the head and neck, bladder, adrenal gland, bone, bone marrow, gall bladder, ganglia, gastrointestinal tract, lung, parathyroid, penis, salivary glands, spleen, stomach, synovial membrane, thymus, uterus, skin, testis and thyroid gland.

In another aspect, the invention provides a method for the treatment of a disorder associated with decreased expression or activity of MTG16 or disorders associated with inactivating mutations in MTG16, comprising administering an isolated DNA molecule as described above to a subject in need of such treatment.

In a further aspect there is provided the use of an isolated DNA molecule as described above in the manufacture of a medicament for the treatment of a disorder associated decreased expression or activity of MTG16 or disorders associated with inactivating mutations in MTG16.

Typically, a vector capable of expressing MTG16 or a fragment or derivative thereof may be administered to a subject that has a decreased expression of MTG16.

Transducing retroviral vectors are often used for somatic cell gene therapy because of their high efficiency of infection and stable integration and expression. The full length MTG16 gene, or portions thereof, can be cloned into a retroviral vector and expression may be driven from its endogenous promoter or from the retroviral long terminal repeat or from a promoter specific for the target cell type of interest. Other viral vectors can be used and include, as is known in the art, adenoviruses, adeno-associated virus, vaccinia virus, papovaviruses, lentiviruses and retroviruses of avian, murine and human origin.

Gene therapy would be carried out according to accepted methods (Friedman, 1991; Culver, 1996). A vector containing a copy of the MTG16 gene linked to expression control elements and capable of replicating inside the cells is prepared. Alternatively the vector may be replication deficient and may require helper cells for replication and use in gene therapy.

Gene transfer using non-viral methods of infection can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes. The gene therapy method of choice must enable production of sufficient protein to provide effective function.

In subjects that express a mutated form of MTG16 it may be possible to prevent malignancy by introducing into the affected cells a wild-type copy of the MTG16 gene such that it recombines with the endogenous mutant gene. This requires a double recombination event for the correction of the gene mutation. Vectors for the introduction of genes in these ways are known in the art, and any suitable vector may be used. Alternatively, introducing another copy of the MTG16 gene bearing a second mutation in that gene may be employed so as to negate the original gene mutation and block any negative effect.

In affected subjects that have decreased expression of MTG16, a mechanism of down-regulation is methylation of the CpG island present in the promoter region of MTG16 and incorporating exon 1b. Therefore, in an alternative approach to therapy, administration of agents that remove MTG16 promoter methylation will reactivate MTG16 gene expression and suppress neoplastic growth of recipient cells.

According to still another aspect of the present invention there is provided a method of treating a disorder associated with decreased expression or activity of MTG16 or disorders associated with inactivating mutations in MTG16, comprising administering a polypeptide, as described above, or an agonist thereof, to a subject in need of such treatment.

In another aspect the invention provides the use of a polypeptide as described above, or an agonist thereof, in the manufacture of a medicament for the treatment of a disorder associated with decreased expression or activity of MTG16 or disorders associated with inactivating mutations in MTG16. Examples of such disorders are described above.

In a further aspect of the invention there is provided a pharmaceutical composition comprising a polypeptide as described above, typically substantially purified MTG16, and a pharmaceutically acceptable carrier may be administered.

The pharmaceutical composition may be administered to a subject to treat or prevent a disorder associated with decreased expression or activity of MTG16 or disorders associated with inactivating mutations in MTG16 including, but not limited to, those provided above. Pharmaceutical compositions in accordance with the present invention are prepared by mixing MTG16 or active fragments or variants thereof having the desired degree of purity, with acceptable carriers, excipients, or stabilizers which are well known. Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

In further embodiments, any of the proteins, agonists or vectors of the invention may be administered in combination with other appropriate therapeutic agents. Selection of the appropriate agents may be made by those skilled in the art, according to conventional pharmaceutical principles. The combination of therapeutic agents may act synergistically to effect the treatment or prevention of the various disorders described above. Using this approach, therapeutic efficacy with lower dosages of each agent may be possible, thus reducing the potential for adverse side effects.

To date, the invention has shown that MTG16 is a tumour suppressor gene whose expression is reduced in cancer cell lines and primary breast tumours. This is likely due to epigenetic mechanisms such as promoter methylation. Loss of functional MTG16 protein within a cell through inactivating mutations in the MTG16 gene may be another mechanism by which cancer develops. In this case, MTG16 polypeptide corresponding to a mutant form of the protein, and cells expressing these, are useful for the screening of candidate pharmaceutical agents in a variety of techniques. Such techniques include, but are not limited to, utilising eukaryotic or prokaryotic host cells that are stably transformed with recombinant polypeptides expressing the mutant polypeptide or fragment, preferably in competitive binding assays. Binding assays will measure for the formation of complexes between mutant MTG16 polypeptide or fragments thereof and the agent being tested, or will measure the degree to which an agent being tested will interfere with the formation of a complex between the mutant MTG16 polypeptide or fragment thereof and a known ligand.

Another technique for drug screening provides high-throughput screening for compounds having suitable binding affinity to the mutant MTG16 polypeptides (see PCT published application WO84/03564). In this stated technique, large numbers of small peptide test compounds can be synthesised on a solid substrate and can be assayed through mutant MTG16 polypeptide binding and washing. Bound mutant MTG16 polypeptide is then detected by methods well known in the art. In a variation of this technique, purified mutant MTG16 polypeptides can be coated directly onto plates to identify interacting test compounds.

An additional method for drug screening involves the use of host eukaryotic cell lines which carry mutations in the MTG16 gene. The host cell lines are also defective at the MTG16 polypeptide level. Other cell lines may be used where MTG16 expression can be switched off. The host cell lines or cells are grown in the presence of various drug compounds and the rate of growth of the host cells is measured to determine if the compound is capable of regulating the growth of MTG16 defective cells.

Mutant MTG16 polypeptides may also be used for screening compounds developed as a result of combinatorial library technology. This provides a way to test a large number of different substances for their ability to modulate activity of a polypeptide. The use of peptide libraries is preferred (see WO 97/02048) with such libraries and their use known in the art.

A substance identified as a modulator of polypeptide function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original binding site. The anti-id could then be used to isolate peptides from chemically or biologically produced peptide banks.

Any of the therapeutic methods described above may be applied to any subject in need of such therapy, including, for example, mammals such as dogs, cats, cows, horses, rabbits, monkeys, and most preferably, humans.

Polynucleotide sequences encoding MTG16 may also be used for the diagnosis of disorders associated with MTG16 tumour suppressor gene function and the use of the DNA molecules of the invention in disorders associated with MTG16 tumour suppressor gene function, or a predisposition to such disorders, is therefore contemplated. Examples of such disorders include, but are not limited to, cancers such as adenocarcinoma, leukaemia, lymphoma, melanoma, myeloma, sarcoma, teratocarcinoma, and, in particular, cancer of the breast, prostate, liver, ovary, neuroectoderm, placenta, skeletal muscle, tonsil, lymph tissue, kidney and colon. Other cancers may include those of the head and neck, bladder, adrenal gland, bone, bone marrow, gall bladder, ganglia, gastrointestinal tract, lung, parathyroid, penis, salivary glands, spleen, stomach, synovial membrane, thymus, uterus, skin, testis and thyroid gland. Such qualitative or quantitative methods are well known in the art.

In another embodiment of the invention, the polynucleotides that may be used for diagnostic purposes include oligonucleotide sequences, genomic DNA and complementary RNA and DNA molecules. The polynucleotides may be used to detect and quantitate gene expression in biopsied tissues in which abnormal expression of MTG16 may be correlated with disease or to detect MTG16 sequence differences between tumour biopsy tissues and normal tissues in which mutations in MTG16 may be correlated with disease. Genomic DNA used for the diagnosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, reverse transcriptase PCR (RT-PCR), hybridization using specific oligonucleotides, restriction enzyme digest and mapping, PCR mapping, RNase protection, and various other methods may be employed. Oligonucleotides specific to particular sequences can be chemically synthesized and labeled radioactively or nonradioactively and hybridized to individual samples immobilized on membranes or other solid-supports or in solution. The presence, absence or excess expression of MTG16 may then be visualized using methods such as autoradiography, fluorometry, or colorimetry.

In a particular aspect, the nucleotide sequences encoding MTG16 may be useful in assays that detect the presence of associated disorders, particularly those mentioned previously. The nucleotide sequences encoding MTG16 may be labelled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding MTG16 in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis of a disorder associated with abnormal expression of MTG16, a normal or standard profile for expression is established. This may be accomplished by combining body fluids or cell extracts taken from normal subjects, either animal or human, with a sequence, or a fragment thereof, encoding MTG16, under conditions suitable for hybridization or amplification. Standard hybridization may be quantified by comparing the values obtained from normal subjects with values from an experiment in which a known amount of a substantially purified polynucleotide is used. Another method to identify a normal or standard profile for expression of MTG16 is through quantitative RT-PCR studies. RNA isolated from body cells of a normal individual, particularly RNA isolated from breast tissue, is reverse transcribed and real-time PCR using oligonucleotides specific for the MTG16 gene is conducted to establish a normal level of expression of the gene. Standard values obtained in both these examples may be compared with values obtained from samples from patients who are symptomatic for a disorder. Deviation from standard values is used to establish the presence of a disorder.

Once the presence of a disorder is established and a treatment protocol is initiated, hybridization assays may be repeated on a regular basis to determine if the level of expression in the patient begins to approximate that which is observed in the normal subject. The results obtained from successive assays may be used to show the efficacy of treatment over a period ranging from several days to months.

In one aspect, hybridization with PCR probes which are capable of detecting polynucleotide sequences, including genomic sequences, encoding MTG16 or closely related molecules may be used to identify nucleic acid sequences which encode MTG16. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding MTG16, allelic variants, or related sequences.

Probes may also be used for the detection of related sequences, and should preferably have at least 50% sequence identity to any of the MTG16 encoding sequences. The hybridization probes of the subject invention may be DNA or RNA and may be derived from the sequence of SEQ ID NO:1 or 2 or from genomic sequences including promoters, enhancers, and introns of the MTG16 gene.

Means for producing specific hybridization probes for DNAs encoding MTG16 include the cloning of polynucleotide sequences encoding MTG16 or MTG16 derivatives into vectors for the production of mRNA probes. Such vectors are known in the art, and are commercially available. Hybridization probes may be labeled by radionuclides such as ³²P or ³⁵S, or by enzymatic labels, such as alkaline phosphatase coupled to the probe via avidin/biotin coupling systems, or other methods known in the art.

According to a further aspect of the invention there is provided the use of a polypeptide as described above in the diagnosis of a disorder associated with MTG16 tumour suppressor gene function, or a predisposition to such disorders.

When a diagnostic assay is to be based upon the MTG16 protein, a variety of approaches are possible. For example, diagnosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind MTG16 may be used for the diagnosis of disorders characterized by abnormal expression of MTG16, or in assays to monitor patients being treated with MTG16 or agonists of MTG16. Antibodies useful for diagnostic purposes may include, but are not limited to, polyclonal, monoclonal, chimeric & single chain antibodies.

For the production of antibodies, various hosts including rabbits, rats, goats, mice, humans, and others may be immunized by injection with MTG16 or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to MTG16 have an amino acid sequence consisting of at least 5 amino acids, and, more preferably, of at least 10 amino acids. It is also preferable that these oligopeptides, peptides, or fragments are identical to a portion of the amino acid sequence of the natural protein and contain the entire amino acid sequence of a small, naturally occurring molecule. Short stretches of MTG16 amino acids may be fused with those of another protein, such as KLH, and antibodies to the chimeric molecule may be produced.

Monoclonal antibodies to MTG16 may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for MTG16 may also be generated. For example, such fragments include, F(ab′)2 fragments produced by pepsin digestion of the antibody molecule and Fab fragments generated by reducing the disulfide bridges of the F(ab′)2 fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between MTG16 and its specific antibody. A two-site, monoclonal-based immunoassay utilizing monoclonal antibodies reactive to two non-interfering MTG16 epitopes is preferred, but a competitive binding assay may also be employed. Diagnostic assays for MTG16 include methods that utilize the antibody and a label to detect MTG16 in human body fluids or in extracts of cells or tissues. The antibodies may be used with or without modification, and may be labelled by covalent or non-covalent attachment of a reporter molecule.

A variety of protocols for measuring MTG16, including ELISAs, RIAs, and flow cytometry of permeabilised cells, are known in the art and provide a basis for diagnosing altered or abnormal levels of MTG16 expression. Normal or standard values for MTG16 expression are established by combining body fluids or cell extracts taken from normal mammalian subjects, preferably human, with antibody to MTG16 under conditions suitable for complex formation. The amount of standard complex formation may be quantitated by various methods, preferably by photometric means. Quantities of MTG16 expressed in subject, control, and disease samples from biopsied tissues are compared with the standard values. Deviation between standard and subject values establishes the parameters for diagnosing disease.

Once an individual has been diagnosed with the disorder, effective treatments can be initiated. These may include administering a selective agonist to the mutant MTG16 so as to restore its function to a normal level or introduction of wild-type MTG16, particularly through gene therapy approaches as described above. Typically, a vector capable of a expressing the appropriate full length MTG16 gene or a fragment of derivative thereof may be administered. In addition, therapies that can reverse the methylation induced transcriptional silencing of the MTG16 gene in affected cells will be useful. In an alternative support approach to therapy, substantially purified MTG16 polypeptide and a pharmaceutically acceptable carrier may be administered as described above.

MTG16, based on its homology to MTG8, is likely to be part of a corepressor complex. MTG16 directs the repressor complex to MTG16 specific interacting proteins leading to transcriptional repression of downstream genes. The MTG16 protein, in its tumour suppressor capacity, may therefore be used in protein interaction studies such as yeast two-hybrid procedures to identify interacting proteins and gene targets. Therefore compounds that are directed to the downstream protein and gene targets of MTG16 may also be of use in therapy. These compounds will act to mimic the function of MTG16 by for example inhibiting MTG16 target gene transcription. Alternatively anti-sense probes or antibodies directed to the MTG16 downstream gene target mRNA or protein respectively may serve to suppress neoplastic growth of target cells.

In further embodiments, complete cDNAs, oligonucleotides or longer fragments derived from any of the polynucleotide sequences described herein may be used as probes in a microarray. The microarray can be used to monitor the expression level of large numbers of genes simultaneously and to identify genetic variants, mutations, and polymorphisms. This information may be used to determine gene function, to understand the genetic basis of a disorder, to diagnose a disorder, and to develop and monitor the activities of therapeutic agents. Microarrays may be prepared, used, and analyzed using methods known in the art. (For example, see Schena et al., 1996; Heller et al., 1997).

The present invention also provides for the production of genetically modified (knock-out, knock-in and transgenic), non-human animal models transformed with the DNA molecules of the invention. These animals are useful for the study of the MTG16 gene function, to study the mechanisms of disease as related to the MTG16 gene, for the screening of candidate pharmaceutical compounds, for the creation of explanted mammalian cell cultures which express the protein or mutant protein and for the evaluation of potential therapeutic interventions.

The MTG16 gene may have been inactivated by knock-out deletion, and knock-out genetically modified non-human animals are therefore provided.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to their relative ease of maintenance and shorter life spans. For certain studies, transgenic yeast or invertebrates may be suitable and preferred because they allow for rapid screening and provide for much easier handling. For longer term studies, non-human primates may be desired due to their similarity with humans.

To create an animal model for mutated MTG16 several methods can be employed. These include generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create a transgenic mouse, which is preferred, a mutant version of MTG16 can be inserted into a mouse germ line using standard techniques of oocyte pronuclear microinjection or transfection or microinjection into embryonic stem cells. Alternatively, if it is desired to inactivate or replace the endogenous MTG16 gene, homologous recombination using embryonic stem cells may be applied.

For oocyte injection, one or more copies of the mutant or wild type MTG16 gene can be inserted into the pronucleus of a just-fertilized mouse oocyte. This oocyte is then reimplanted into a pseudo-pregnant foster mother. The liveborn mice can then be screened for integrants using analysis of tail DNA for the presence of human MTG16 gene sequences. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

According to still another aspect of the invention there is provided the use of genetically modified non-human animals for the screening of candidate pharmaceutical compounds.

In a still further aspect of the invention there is provided a nucleic acid encoding a mutant MTG16 polypeptide which cannot form a complex with a wild-type protein with which wild-type MTG16 does form a complex.

According to a still further aspect of the invention there is provided a mutant MTG16 polypeptide which cannot form a complex with a wild-type protein with which wild-type MTG16 does form a complex.

In a still further aspect of the present invention there is provided the use of a complex as described above in screening for candidate pharmaceutical compounds.

It will be clearly understood that, although a number of prior art publications are referred to herein, this reference does not constitute an admission that any of these documents forms part of the common general knowledge in the art, in Australia or in any other country. Throughout this specification and the claims, the words “comprise”, “comprises” and “comprising” are used in a non-exclusive sense, except where the context requires otherwise.

### BRIEF DESCRIPTION OF THE DRAWINGS

FIG. 1. Schematic representation of tumours with interstitial and terminal allelic loss on chromosome arm 16q in the two series of tumour samples. Polymorphic markers are listed according to their order on 16q from centromere to telomere and the markers used for each series are indicated by X. Tumour identification numbers are shown at the top of each column. At the right of the figure, the three smallest regions of loss of heterozygosity are indicated.

FIG. 2. Semi-quantitative RT-PCR analysis of MTG16. A: Primers for PCR were specific for the 3′ UTR of MTG16. Products were run on a 2.5% agarose gel and the expected amplicon size is indicated by an arrow. M: DNA size markers; 1: Foetal brain; 2: Normal mammary gland; 3: MCF12A; 4: BT549; 5: MDA-MB-468; 6: CAMA-1; 7: ZR75-30; 8: MDA-MB-157; 9: MDA-MB-134; 10: ZR75-1; 11: SKBR3; 12: MDA-MB-231; 13: T47D; 14: MDA-MB-436; 15: PC3: g: Genomic DNA; n: No DNA template: +: Reverse transcription reaction included reverse transcriptase; −: Reverse transcription reaction did not include reverse transcriptase. Results indicate decreased expression of MTG16 in the breast cancer cell lines BT549, MDA-MB468, MDA-MB-157 and MDA-MB-231 as well as the prostate cancer cell line PC3. Little or no expression was observed in SKBR3. B: Control RT-PCR using primers specific for the house-keeping gene Esterase D. Results indicate all control and cell line reverse transcription reactions were successful. All primers used for semi-quantitative PCR are shown in Table 2.

FIG. 3. Quantitative RT-PCR expression analysis of control house-keeping genes in breast cancer cell lines, a prostate cancer cell line and normal control tissues. The degree of variation in mRNA expression levels for Cyclophilin, RNA polymerase II subunit and APRT following normalisation of cDNA templates is shown. Amplicon copy numbers in normalized normal mammary gland (breast) cDNA were arbitrarily set to a ‘baseline’ of 1.0e+06 copies (empty bar). Breast cancer cell lines and other normal tissue cDNA copy numbers were calculated relative to the ‘baseline’. Grey filled bars represent amplicon fold expression down-regulation compared to the baseline reference, while black filled bars represent amplicon fold expression up-regulation from the baseline reference. Three way combinations for normalisation between these house-keeping genes demonstrate a mean 7-fold and maximum 50-fold variance in mRNA expression level between samples.

FIG. 4. Quantitative RT-PCR expression analysis of the Esterase D gene in cell lines and normal control tissues. Cycle number is indicated on the x axis while the y axis indicates relative fluorescence. The RotorGene 2000 output indicates successful normalisation of cDNA templates.

FIG. 5. Quantitative RT-PCR expression analysis of the MTG16 gene in cell lines and normal control tissues. Cycle number is indicated on the x axis while the y axis indicates relative fluorescence. The RotorGene 2000 output indicates that breast cancer cell lines MDA-MB-468, MDA-MB-157, BT549, SKBR3 and MDA-MB-231 show reduced expression when compared to fetal brain and normal mammary gland control tissues.

FIG. 6. A summary of fold differences in expression of breast cancer cell lines compared with normal breast tissue measured with quantitative RT-PCR using MTG16 specific primers. As previously, MTG16 copy numbers in normalized normal mammary gland (breast) cDNA were arbitrarily set to a ‘baseline’ of 1.0e+06 copies (empty bar). Breast cancer cell lines and other normal tissue cDNA copy numbers were calculated relative to the ‘baseline’. Grey filled bars represent amplicon fold expression down-regulation compared to the baseline reference, while black filled bars represent amplicon fold expression up-regulation from the baseline reference. A significant reduction in expression of the MTG16 gene was observed in breast cancer cell lines MDA-MB-468, MDA-MB-157, BT549, SKBR3 and MDA-MB-231. This data confirms the reduced expression of MTG16 in these cell lines observed with semi-quantitative RT-PCR analysis.

FIG. 7. In situ hybridisation of a primary tumour breast tissue section with anti-sense MTG16 probe. A: A region of the tumour tissue section in which normal breast epithelial cells are present. The top panel shows a low power (×200) view of normal mammary ducts which are lined by epithelial cells, each of which is staining positively for MTG16 mRNA. The bottom panel is a high power (×1000) view of a single normal duct which highlights the presence of MTG16 mRNA in the nucleus and cytoplasm of each epithelial cell. B: A region of the same tissue section slide in which tumour cells are present. The top panel is a low power (×200) view of tumour cell masses that show extremely reduced MTG16 mRNA staining. In the high power (×1000) view (bottom panel) individual tumour cells can be seen. The anti-sense probe detected very poor expression of MTG16 mRNA in the tumour cells. An inflammatory infiltrate is highlighted which shows positive expression for MTG16. All positive and negative control experiments conducted subsequently supported these findings.

FIG. 8. Expression of MTG16 in breast cancer cell lines. SK-BR-3, MDA-MB 231 and MDA-MB 468 breast cancer cell lines were infected with recombinant retroviruses expressing Myc-tagged MTG16 or Neo only (empty vector) RNA. Two days after infection G418 was added to the cell medium and two weeks later surviving colonies were fixed, stained with Giemsa and counted. A: Data represent results from at least two independent experiments. The values shown are the mean and range of duplicate samples. B: Photographs of colonies from representative plates for each cell line expressing empty vector (top panel) or recombinant MTG16 (bottom panel). This figure indicates that re-expression of MTG16 in breast cancer cell lines that show reduced expression of MTG16 is able to reduce the growth of the cancer cells.

FIG. 9. Cell localisation studies of the MTG16 protein. GFP-tagged MTG16 was found to produce a distinct punctate pattern over weaker diffuse staining in the cell nuclei (FIG. 9A) compared to even cytoplasmic and nuclear distribution of the GFP alone (FIG. 9B).

FIG. 10. MTG16 transcriptional regulation. 293T cells were co-transfected with 1 μg CAT reporter plasmid and increasing amounts (0.3-3 μg) of pMMTG16 expressing MTG16 fused to the GAL4 DNA-binding domain (DBD). GAL4 DBD only was used as a negative control and the NK-10 repressor domain expressing plasmid was used as a positive control. The cells were harvested 24 hours post transfection. CAT concentration was determined by ELISA and normalised to β-galactosidase activity from the pcDNA3-β-gal vector which was used as an internal control for transfection efficiency. The data shown are mean±SEM from triplicate samples representative of two independent experiments.

### MODES FOR PERFORMING THE INVENTION

### EXAMPLE 1

### Collection of Breast Cancer Patient Material

Two series of breast cancer patients were analysed for this study. Histopathological classification of each tumour specimen was carried out by our collaborators according to World Health Organisation criteria (WHO, 1981). Patients were graded histopathologically according to the modified Bloom and Richardson method (Elston and Ellis, 1990) and patient material was obtained upon approval of local Medical Ethics Committees. Tumour tissue DNA and peripheral blood DNA from the same individual was isolated as previously described (Devilee et al., 1991) using standard laboratory protocols.

Series 1 consisted of 189 patients operated on between 1986 and 1993 in three Dutch hospitals, a Dutch University and two peripheral centres. Tumour tissue was snap frozen within a few hours of resection. For DNA isolation, a tissue block was selected only if it contained at least 50% of tumour cells following examination of haematoxilin and eosin stained tissue sections by a pathologist. Tissue blocks that contained fewer than 50% of tumour cells were omitted from further analysis.

Series 2 consisted of 123 patients operated on between 1987 and 1997 at the Flinders Medical Centre in Adelaide, Australia. Of these, 87 were collected as fresh specimens within a few hours of surgical resection, confirmed as malignant tissue by pathological analysis, snap frozen in liquid nitrogen, and stored at −70° C. The remaining 36 tumour tissue samples were obtained from archival paraffin embedded tumour blocks. Prior to DNA isolation, tumour cells were microdissected from tissue sections mounted on glass slides so as to yield at least 80% tumour cells. In some instances, no peripheral blood was available such that pathologically identified paraffin embedded non-malignant lymph node tissue was used instead.

### EXAMPLE 2

### LOH Analysis of Chromosome 16q Markers in Breast Cancer Samples

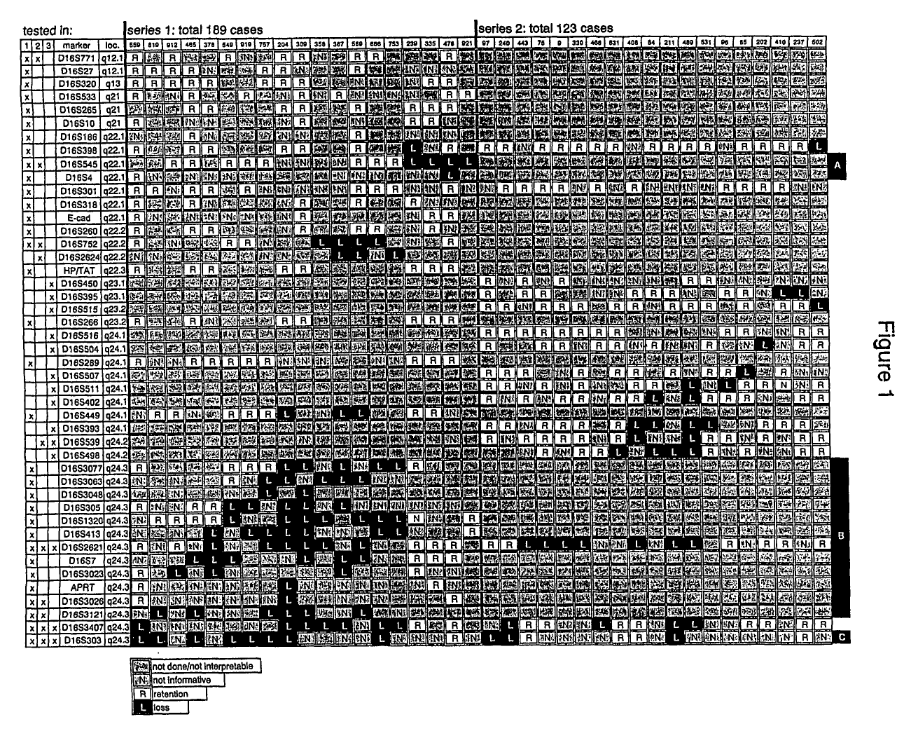

A total of 45 genetic markers were used for the LOH analysis of breast tumour and matched normal DNA samples. FIG. 1 indicates for which tumour series they were used and their cytogenetic location. Details regarding all markers can be obtained from the Genome Database (GDB) at http://www.gdb.org. The physical order of markers with respect to each other was determined from a combination of information in GDB, by mapping on a chromosome 16 somatic cell hybrid map (Callen et al., 1995) and by genomic sequence information.

Four alternative methods were used for the LOH analysis:
1) For RFLP and VNTR markers, Southern blotting was used to test for allelic imbalance. These markers were used on only a subset of samples. Methods used were as previously described (Devilee et al., 1991).
2) Microsatellite markers were amplified from tumour and normal DNA using the polymerase chain reaction (PCR) incorporating standard methodologies (Weber and May, 1989; Sambrook et al., 1989). A typical reaction consisted of 12 ul and contained 100 ng of template, 5 pmol of both primers, 0.2 mM of each dNTP, 1 uCurie [α-³²P]dCTP, 1.5 mM MgCl₂, 1.2 ul Supertaq buffer and 0.06 units of Supertaq (HT biotechnologies). A Phosphor Imager type 445 SI (Molecular Dynamics, Sunnyvale, Calif.) was used to quantify ambiguous results. In these cases, the Allelic Imbalance Factor (AIF) was determined as the quotient of the peak height ratios from the normal and tumour DNA pair. The threshold for allelic imbalance was defined as a 40% reduction of one allele, agreeing with an AIF of ≧1.7 or ≦0.59. This threshold is in accordance with the selection of tumour tissue blocks containing at least 50% tumour cells with a 10% error-range. The threshold for retention has been previously determined to range from 0.76 to 1.3 (Devilee et al., 1994). This leaves a range of AIFs (0.58-0.75 and 1.31-1.69) for which no definite decision has been made. This “grey area” is indicated by grey boxes in FIG. 1 and tumours with only “grey area” values were discarded completely from the analysis.
3) The third method for determining allelic imbalance was similar to the second method above, however radioactively labelled dCTP was omitted. Instead, PCR of polymorphic microsatellite markers was done with one of the PCR primers labelled fluorescently with FAM, TET or HEX. Analysis of PCR products generated was on an ABI 377 automatic sequencer (PE Biosystems) using 6% polyacrylamide gels containing 8M urea. Peak height values and peak sizes were analysed with the GeneScan programme (PE Biosystems). The same thresholds for allelic imbalance, retention and grey areas were used as for the radioactive analysis.
4) An alternative fluorescent based system was also used. In this instance PCR primers were labelled with fluorescein or hexachlorofluorescein. PCR reaction volumes were 20 ul and included 100 ng of template, 100 ng of each primer, 0.2 mM of each dNTP, 1-2 mM MgCl₂, 1× AmpliTaq Gold buffer and 0.8 units AmpliTaq Gold enzyme (Perkin Elmer). Cycling conditions were 10 cycles of 94° C. for 30 seconds, 60° C. for 30 seconds, 72° C. for 1 minute, followed by 25 cycles of 94° C. 30 seconds, 55° C. for 30 seconds, 72° C. for 1 minute, with a final extension of 72° C. for 10 minutes. PCR amplimers were analysed on an ABI 373 automated sequencer (PE Biosystems) using the GeneScan programme (PE Biosystems). The threshold range of AIF for allele retention was defined as 0.61-1.69, allelic loss as ≦0.5 or ≧2.0, or the “grey area” as 051-0.6 or 1.7-1.99.

The first three methods were applied to the first tumour series while the last method was adopted for the second series of tumour samples. For statistical analysis, a comparison of allelic imbalance data for validation of the different detection methods and of the different tumour series was done using the Chi-square test.

The identification of the smallest region of overlap (SRO) involved in LOH is instrumental for narrowing down the location of a putative tumour suppressor gene targeted by LOH. FIG. 1 shows the LOH results for tumour samples, which displayed small regions of loss (ie interstitial and telomeric LOH) and does not include samples that showed complex LOH (alternating loss and retention of markers). When comparing the two sample sets at least three consistent regions emerge with two being at the telomere in band 16q24.3 and one at 16q22.1. The region at 16q22.1 is defined by the markers D16S398 and D16S301 and is based on the interstitial LOH events seen in three tumours from series 1 (239/335/478) and one tumour from series 2 (237). At the telomere (16q24.2-16q24.3), the first region is defined by the markers D16S498 and D16S3407 and is based on four tumours from series 2 (443/75/631/408) while the second region (16q24.3) extends from D16S3407 to the telomere and is based on one tumour from series 1 (559) and three from series 2 (97/240/466). LOH limited to the telomere but involving both of the regions identified at this site could be found in an additional 17 tumour samples.

Other studies have shown that the long arm of chromosome 16 is also a target for LOH in prostate, lung, hepatocellular, ovarian, primitive neuroectodermal and Wilms' tumours. Detailed analysis of prostate carcinomas has revealed an overlap in the smallest regions of LOH seen in this cancer to that seen with breast cancer which suggests that 16q harbours a multi-tumour suppressor gene.

### EXAMPLE 3

### Construction of a Physical Map of 16q24.3

To identify novel candidate tumour suppressor genes mapping to the smallest regions of overlap at 16q24.3, a clone based physical map contig covering this region was needed. At the start of this phase of the project the most commonly used and readily accessible cloned genomic DNA fragments were contained in lambda, cosmid or YAC vectors. During the construction of whole-chromosome 16 physical maps, clones from a number of YAC libraries were incorporated into the map (Doggett et al., 1995). These included clones from a flow-sorted chromosome 16-specific YAC library (McCormick et al., 1993), from the CEPH Mark I and MegaYAC libraries and from a half-telomere YAC library (Riethman et al., 1989). Detailed STS and Southern analysis of YAC clones mapping at 16q24.3 established that very few were localised between the CY2/CY3 somatic cell hybrid breakpoint and the long arm telomere. However, those that were located in this region gave inconsistent mapping results and were suspected to be rearranged or deleted. Coupled with the fact that YAC clones make poor sequencing substrates, and the difficulty in isolating the cloned human DNA, a physical map based on cosmid clones was the initial preferred option.

A flow-sorted chromosome 16 specific cosmid library had previously been constructed (Longmire et al., 1993), with individual cosmid clones gridded in high-density arrays onto nylon membranes. These filters collectively contained ˜15,000 clones representing an approximately 5.5 fold coverage of chromosome 16. Individual cosmids mapping to the critical regions at 16q24.3 were identified by the hybridisation of these membranes with markers identified by this and previous studies to map to the region. The strategy to align overlapping cosmid clones was based on their STS content and restriction endonuclease digestion pattern. Those clones extending furthest within each initial contig were then used to walk along the chromosome by the hybridisation of the ends of these cosmids back to the high-density cosmid grids. This process continued until all initial contigs were linked and therefore the region defining the location of the breast cancer tumour suppressor genes would be contained within the map. Individual cosmid clones representing a minimum tiling path in the contig were then used for the identification of transcribed sequences by techniques such as exon trapping and genomic sequencing.

Chromosome 16 was sorted from the mouse/human somatic cell hybrid CY18, which contains this chromosome as the only human DNA, and Sau3A partially digested CY18 DNA was ligated into the BamHI cloning site of the cosmid sCOS-1 vector. All grids were hybridised and washed using methods described in Longmire et al. (1993). Briefly, the 10 filters were pre-hybridised in 2 large bottles for at least 2 hours in 20 ml of a solution containing 6×SSC; 10 mM EDTA (pH8.0); 10× Denhardt's; 1% SDS and 100 μg/ml denatured fragmented salmon sperm DNA at 65° C. Overnight hybridisations with [α-³²P]dCTP labelled probes were performed in 20 ml of fresh hybridisation solution at 65° C. Filters were washed sequentially in solutions of 2×SSC; 0.1% SDS (rinse at room temperature), 2×SSC; 0.1% SDS (room temperature for 15 minutes), 0.1×SSC; 0.1% SDS (room temperature for 15 minutes), and 0.1×SSC; 0.1% SDS (twice for 30 minutes at 50° C. if needed). Membranes were exposed at −70° C. for between 1 to 7 days.

Initial markers used for cosmid grid screening were those known to be located below the somatic cell hybrid breakpoints CY2/CY3 and the long arm telomere (Callen et al., 1995). These included three genes, CMAR, DPEP1, and MC1R; the microsatellite marker D16S303; an end fragment from the cosmid 317E5, which contains the BBC1 gene; and four cDNA clones, yc81e09, yh09a04, D16S532E, and ScDNA-C113. The IMAGE consortium cDNA clone, yc81e09, was obtained through screening an arrayed normalised infant brain oligo-dT primed cDNA library (Soares et al., 1994), with the insert from cDNA clone ScDNA-A55. Both the ScDNA-A55 and ScDNA-C113 clones were originally isolated from a hexamer primed heteronuclear cDNA library constructed from the mouse/human somatic cell hybrid CY18 (Whitmore et al., 1994). The IMAGE cDNA clone yh09a04 was identified from direct cDNA selection of the cosmid 37B2 which was previously shown to map between the CY18A(D2) breakpoint and the 16q telomere. The EST, D16S532E, was also mapped to the same region. Subsequent to these initial screenings, restriction fragments representing the ends of cosmids were used to identify additional overlapping clones.

Contig assembly was based on methods previously described (Whitmore et al., 1998). Later during the physical map construction, genomic libraries cloned into BAC or PAC vectors (Genome Systems or Rosewell Park Cancer Institute) became available. These libraries were screened to aid in chromosome walking or when gaps that could not be bridged by using the cosmid filters were encountered. All BAC and PAC filters were hybridised and washed according to manufacturers recommendations. Initially, membranes were individually pre-hybridised in large glass bottles for at least 2 hours in 20 ml of 6×SSC; 0.5% SDS; 5× Denhardt's; 100 μg/ml denatured salmon sperm DNA at 65° C. Overnight hybridisations with [α-³²P]dCTP labelled probes were performed at 65° C. in 20 ml of a solution containing 6×SSC; 0.5% SDS; 100 μg/ml denatured salmon sperm DNA. Filters were washed sequentially in solutions of 2×SSC; 0.5% SDS (room temperature 5 minutes), 2×SSC; 0.1% SDS (room temperature 15 minutes) and 0.1×SSC; 0.5% SDS (37° C. 1 hour if needed). PAC or BAC clones identified were aligned to the existing contig based on their restriction enzyme pattern or formed unique contigs which were extended by additional filter screens.

As the microsatellite D16S303 was known to be the most telomeric marker in the 16q24.3 region (Callen et al., 1995), fluorescence in situ hybridisation (FISH) to normal metaphase chromosomes using whole cosmids mapping in the vicinity of this marker, was used to define the telomeric limit for the contig. Whole cosmid DNA was nick translated with biotin-14-dATP and hybridised in situ at a final concentration of 20 ng/μl to metaphases from 2 normal males. The FISH method had been modified from that previously described (Callen et al., 1990). Chromosomes were stained before analysis with both propidium iodide (as counter-stain) and DAPI (for chromosome identification). Images of metaphase preparations were captured by a cooled CCD camera using the CytoVision Ultra image collection and enhancement system (Applied Imaging Int. Ltd.). The cosmid 369E1 showed clear fluorescent signals at the telomere of the long arm of chromosome 16. However, this probe also gave clear signal at the telomeres of chromosomal arms 3q, 7p, 9q, 11p, and 17p. Conversely, the cosmid 439G8, which mapped proximal to D16S303, gave fluorescent signals only at 16qter with no consistent signal detected at other telomeres. These results enabled us to establish the microsatellite marker D16S303 as the boundary of the transition from euchromatin to the subtelomeric repeats, providing a telomeric limit to the contig.

A high-density physical map consisting of cosmid, BAC and PAC clones has been established, which extends approximately 3 Mb from the telomere of the long arm of chromosome 16. This contig extends beyond the CY2/CY3 somatic cell hybrid breakpoint and includes the 2 regions of minimal LOH identified at the 16q24.3 region in breast cancer samples. To date, a single gap of unknown size exists in the contig and will be closed by additional contig extension experiments. The depth of coverage has allowed the identification of a minimal tiling path of clones which were subsequently used as templates for gene identification methods such as exon trapping and genomic DNA sequencing.

### EXAMPLE 4

### Identification of Candidate Tumour Suppressor Genes by Analysis of Genomic DNA Sequence

Selected minimal overlapping BAC and PAC clones from the physical map contig were sequenced in order to aid in the identification of candidate tumour suppressor genes. DNA was prepared from selected clones using a large scale DNA isolation kit (Qiagen). Approximately 25-50 ug of DNA was then sheared by nebulisation (10 psi for 45 seconds) and blunt ended using standard methodologies (Sambrook et al., 1989). Samples were then run on an agarose gel in order to isolate DNA in the 2-4 Kb size range. These fragments were cleaned from the agarose using QIAquick columns (Qiagen), ligated into puc18 and used to transform competent XL-1 Blue E. coli cells. DNA was isolated from transformed clones and was sequenced using vector specific primers on an ABI377 sequencer. Analysis of genomic sequence was performed using PHRED, PHRAP and GAP4 software on a SUN workstation. To assist in the generation of large contigs of genomic sequence, information present in the htgs database at NCBI (National Centre for Biotechnology Information) was incorporated into the assembly phase of the sequence analysis. The resultant genomic sequence contigs were masked for repeats and analysed using the BLAST algorithm (Altschul et al., 1997) to identify nucleotide and protein sequence homology to sequences in the NCBI non-redundant and EST databases. The genomic sequence was also analysed for predicted gene structure using the GENSCAN program.

Homologous IMAGE Consortium cDNA clones were purchased from Genome Systems and were sequenced. These longer stretches of sequence were then compared to known genes by nucleotide and amino acid sequence comparisons using the above procedures. Any sequences that are expressed in the breast are considered to be candidate tumour suppressor genes. Those genes whose function could implicate it in the tumourigenic process, as predicted from homology searches with known proteins, were treated as the most likely candidates. Evidence that a particular candidate is the responsible gene comes from the identification of defective alleles of the gene in affected individuals or from analysis of the expression levels of a particular candidate gene in breast cancer samples compared with normal control tissues.

### EXAMPLE 5

### Identification of the MTG16 Gene

Sequence analysis of BAC830F9 indicated the presence of a number of transcribed sequences. One of these was the MTG16 gene. This gene had previously been mapped to chromosome 16q24 (Gamou et al., 1998) however this study has provided a precise localisation of the gene to a particular BAC clone in the 16q24.3 region. Further, this study has shown that MTG16 lies in a region of minimal LOH seen in breast and prostate cancers and is therefore a candidate tumour suppressor gene.

MTG16 is a member of the MTG8 (ETO) family of proteins. Both MTG8 and MTG16 are involved in independent translocations with the AML1 gene forming rare but recurrent chromosomal abnormalities associated with myeloid malignancies (Miyoshi et al., 1991; Gamou et al., 1998). These translocations result in the formation of novel fusion proteins which are critical in the development of the leukaemia.

While no functional information is known about MTG16, MTG8 has been extensively characterised. MTG8 encodes a protein with two putative zinc fingers and several proline rich regions and is presumed to function as a transcription factor. This gene shows strong homology to the Drosophila nervy gene, especially in four regions named nervy homology regions (NHR1-4). The NHR4 region contains the two zinc finger motifs which have been reported to be essential for the interaction with the N—CoR protein (Wang et al., 1998). N—CoR has been shown to form a complex with mammalian Sin3 and histone deacetylase 1 (HDAC1) that alters chromatin structure and mediates transcriptional repression by nuclear receptors and by a number of oncoregulatory proteins (Heinzel et al., 1997; Alland et al., 1997). Subsequently, MTG8, through its interaction with the N—CoR/mSin3/HDAC1 complex, has been shown to be a potent repressor of transcription (Wang et al., 1998).

In the AML1/MTG8 translocation product associated with myeloid malignancies, the transactivation domain of the AML1 gene, which would normally bind to the transcriptional coactivators p300/CBP, is replaced by almost the entire MTG8 protein. This fusion protein therefore recruits a corepressor complex containing HDAC activity instead of the co-activators p300/CBP to AML1 responsive genes giving rise to leukaemia.

Despite the insight into the function of the oncogene AML1/MTG8, the precise normal physiological role of MTG8 is not yet clear, because it does not show DNA binding activity. However it has been shown to potentiate transcriptional repression induced by other transcription factors, such as the promyelocytic leukemia zinc finger protein, by recruiting corepressors and histone deacetylase (Melnick et al., 2000).

MTG16 has a high degree of homology to MTG8 and also contains the four NHR regions. It is reasonable to assume therefore that MTG16 could also be able to repress transcription of genes through an interaction with a corepressor complex such as the N—CoR/mSin3/HDAC1 complex or a similar complex.

### EXAMPLE 6

### Characteristics of the MTG16 Gene

The sequence and genomic structure of MTG16 has been reported elsewhere (Gamou et al., 1998).

MTG16 exists as two isoforms (MTG16a and MTG16b) due to the alternate splicing of exon 3 (present in MTG16a only) and the use of separate first exons. Analysis of the genomic sequence identified 5′ to exon 1a by the applicants indicates the continuation of the open reading frame beyond the originally proposed methionine start codon (Gamou et al., 1998). This provides an additional 177 amino acids before an in-frame stop codon is identified. The previously reported genomic structure of MTG16 was confirmed (Gamou et al., 1998), however the precise location of exon 1a was determined and intron sizes were now able to be defined precisely (Table 1). The presence of a CpG island incorporating and extending 5′ to exon 1b was also identified.

A BLASTN search of the human EST database at NCBI revealed matches to a number of cDNA clones, corresponding to the UniGene cluster Hs.110099 (http://www.ncbi.nlm.nih.gov/UniGene/clust.cgi?ORG=Hs&CID=110099). The clones in this cluster have been isolated from B-cells, blood, brain, cervix, colon, eye, kidney tumour, lymph, marrow, muscle, pancreas, placenta and tonsil tissue, indicating that the MTG16 gene is expressed in a wide variety of tissues. In addition, expression studies of MTG16 (see below) indicate the gene is also present in breast tissue.

Both isoforms of MTG16 share significant homology to the MTG8 gene (67% and 75% identity respectively) and another member of the family, MTGR1 (54% and 61% identity respectively). Due to the high homology of MTG16 to MTG8 and the conservation of the NHR1-4 regions between the two genes, we proposed that MTG16 is a candidate tumour suppressor gene at the 16q24.3 region. To test for inactivating mechanisms of the gene in breast and other cancers, expression and mutation analysis studies were initiated.

### EXAMPLE 7

### Examination of the Expression Level of MTG16 in Breast Cancer Cell Lines

To investigate a potential role for MTG16 in breast cancer, the level of expression of the gene in breast cancer cell lines was compared with normal tissue controls. Examination of the genomic sequence surrounding MTG16b shows that the 5′ end including exon 1b is extremely G-C rich suggesting the presence of a CpG island. While not wishing to be bound by theory, this raises the possibility that epigenetic mechanisms to inactivate MTG16b isoform function may exist. Abnormal methylation at this site may result in a down-regulation of MTG16b transcription of the remaining copy of the gene. Recent studies have shown that this mechanism has been responsible for the inactivation of other tumour suppressor genes such as RB1 (Ohtani-Fujita et al., 1997), VHL (Prowse et al., 1997), MLH1 (Herman et al., 1998) and BRCA1 (Esteller et al., 2000).

To detect the level of expression of MTG16 in cancer samples compared with normal controls, both semi-quantitative and quantitative RT-PCR using MTG16 specific primers was done. This initially involved the isolation of RNA from breast cancer cell lines along with appropriate cell line controls.

Breast/Prostate Cancer Cell Lines and RNA Extraction

The breast cancer cell lines BT549, MDA-MB-468, CAMA-1, MDA-MB-134, ZR75-1, ZR75-30, MDA-MB-157, ZR75-1, SKBR3, MDA-MB-231, T47D, and MDA-MB-436 were purchased from ATCC (USA) along with the normal breast epithelial cell line MCF12A and the prostate cancer cell line PC3. Cell lines were cultured to 80% confluency in RPMI+FCS or OPTI-MEM media at 37° C. in air supplemented with 5% CO₂. Detached cells were washed thoroughly, resuspended in PBS and pelleted by centrifugation at 1,200×g for 5 minutes. Breast cancer cell lines were chosen for RT-PCR analysis that demonstrated homozygosity for a number of markers mapping to chromosome 16q indicating potential LOH for this chromosomal arm (Callen et al., 2001). Total RNA was extracted using the RNAeasy kit (Qiagen) or the TRIzol™ reagent (Gibco BRL) according to manufacturers recommendations. PolyA⁺ mRNA was subsequently isolated from all sources using the Oligotex bead system (Qiagen). PolyA⁺ mRNA from normal mammary gland, prostate, ovary and liver was purchased commercially (Clontech, USA).

Control human mammary epithelial cells (HMEC) were purchased from Clonetics (San Diego) and cultured in serum free media supplied by the manufacturer. Total RNA from these cells was extracted using the Trizol reagent (Gibco BRL) according to manufacturers recommendations.

Reverse Transcription

Total RNA and PolyA⁺ mRNA was primed with oligo-dT primers and reverse transcribed using the Omniscript RT kit (Qiagen) according to manufacturers conditions or using Superscript™ RNaseH⁻ reverse transcriptase (Gibco BRL). In the latter method, 1 μg of total RNA sample was mixed with 500 ng of oligo (dT)₁₆ and made up to a volume of 10 μl with DEPC treated water. Following a 10 minute incubation at 70° C., 4 μl of 5× first strand buffer, 2 μl of 0.1 M DTT, 1 μl of 10 mM dNTP, 20 units of RNAsin™ (Promega) and 100 units of Superscript reverse transcriptase were added and the reaction incubated at 42° C. for 2 hours. Reactions were terminated at 95° C. for 5 minutes and cDNA:RNA hybrids were removed from samples by addition of 2 units of RNase H (Promega) and incubation at 37° C. for 30 minutes. Control reactions were included for each RNA template, which omitted reverse transcriptase from the cDNA synthesis step. This was to determine the presence of any genomic DNA contamination in the RNA samples. All samples were stored at −20° C.

Semi-Quantitative RT-PCR

First strand cDNA synthesised was PCR amplified with primers specific for the MTG16 3′ untranslated region using the HotStarTaq kit (Qiagen) in a 10 ul reaction volume for 35 cycles. Initially, primers to the control housekeeping gene Esterase D were used in a separate reaction to confirm the presence of cDNA templates for each reverse transcription reaction. MTG16 and Esterase D primer sequences used are listed in Table 2 and are represented by the SEQ ID Numbers: 5-8. All PCR products were analysed on agarose gels and visualised with ethidium bromide staining.

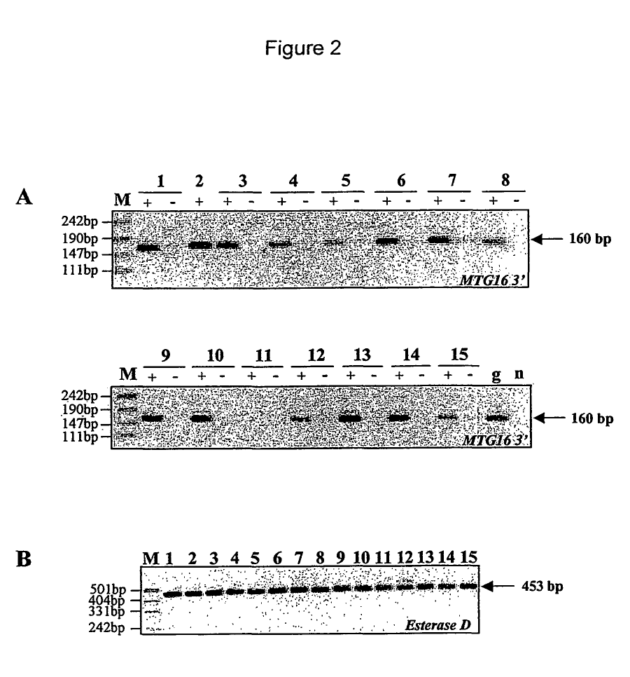

FIG. 2 shows the results of the semi-quantitative RT-PCR reactions. As the Esterase D control primers indicate, cDNA synthesis from all template samples was successful. However, while normal fetal brain and mammary gland samples showed strong expression of the MTG16 gene, differential expression was observed in a number of cancer cell lines. Poor expression of MTG16 was seen in the breast cancer cell lines BT-549, MDA-MB-468, MDA-MB-157, and MDA-MB-231, while little or no expression was observed in SKBR3. Poor expression was also seen in the prostate cancer cell line PC3. These results were reproducible.

Quantitative RT-PCR

Generation of Internal Standard Curve Amplicons

All real-time amplicons were generated with primers designed by Lasergene Primer Select™ (DNASTAR) within an average maximum of 1 kb from the transcript 3′ end. Internal standard curve amplicons were generated from a mixed pool of normal tissue cDNA using the HotStarTaq™ DNA Polymerase kit (Qiagen). A reaction mix sufficient to generate >1 μg of amplicon cDNA contained 10 μl of 10× PCR buffer (containing 15 mM MgCl₂), 2 μl of 10 mM dNTP mix, 0.5 μM of each primer, 0.5 μl of 2.5 units HotStarTaq polymerase (Qiagen), 100 ng of cDNA template and DEPC treated water to 100 μl. Amplification cycling was performed as follows: 94° C. for 10 minutes followed by 35 cycles at 93° C. for 20 seconds, 60° C. for 30 seconds and 70° C. for 30 seconds with a final extension at 72° C. for 4 minutes. Amplicons were purified using the QIAquick gel extraction kit (Qiagen) according to manufacturers conditions and concentrations were measured at A₂₆₀. Purified amplicons were serially diluted 10-fold from 10 ng/μl to 1 fg/μl. These dilutions served as internal standards of known concentration for real-time analysis of MTG16 specific amplicons as described below.

Real-Time PCR

All cDNA templates were amplified using the SYBR Green I PCR Master Mix kit (PE Biosystems, USA). PCR reactions included 12.5 ul of SYBR Green I PCR Master mix, 0.2 μM of each primer, 30 ng of cDNA template (approximately 2 ul) and DEPC treated water to 25 ul. Real-time PCR analysis was performed using the Rotor-Gene™2000 (Corbett Research, AUS) with the following amplification cycling conditions: 94° C. for 10 minutes followed by 45 cycles of 93° C. for 20 seconds, 60° C. for 30 seconds and 70° C. for 30 seconds. Fluorescence data was acquired at 510 nm during the 72° C. extension phase. Melt curve analyses were performed with an initial 99-50° C. cycling followed by fluorescence monitoring during heating at 0.2° C./second to 99° C. Prior to real-time quantification, product size and specificity was confirmed by ethidium bromide staining of 2.5% agarose gels following electrophoresis of completed PCRs. Control and MTG16 specific primers used for all real-time PCR applications are listed in Table 2 and are represented by the SEQ ID Numbers: 7-16.

Real-Time PCR Quantification

Quantification analyses were performed on the Rotor-Gene™ DNA sample analysis system (Version 4.2, Build 96). Standard curves were generated by amplifying 10-fold serial dilutions (1 μl of 10 ρg/μl down to 1 μl of 1 fg/μl in triplicate) of the internal standard amplicon during real-time PCR of MTG16 amplicons from normal tissues and breast cancer cell lines. Internal standard amplicon concentrations were arbitrarily set to 1.0e+12 copies for 10 ρg standards to 1.0e+08 copies for 1 fg standards. C_{T} (cycle threshold) coefficients of variation for all internal standard dilutions averaged 2% between triplicate samples within the same and different runs. The Rotor-Gene™ quantification software generated a line of best-fit at the parameter C_{T} and determined unknown normal tissue and breast cancer cell line MTG16 amplicon copy numbers by interpolating the noise-band intercept of MTG16 amplicons against the internal standards with known copy numbers.

Normalization and Relative Expression of Data

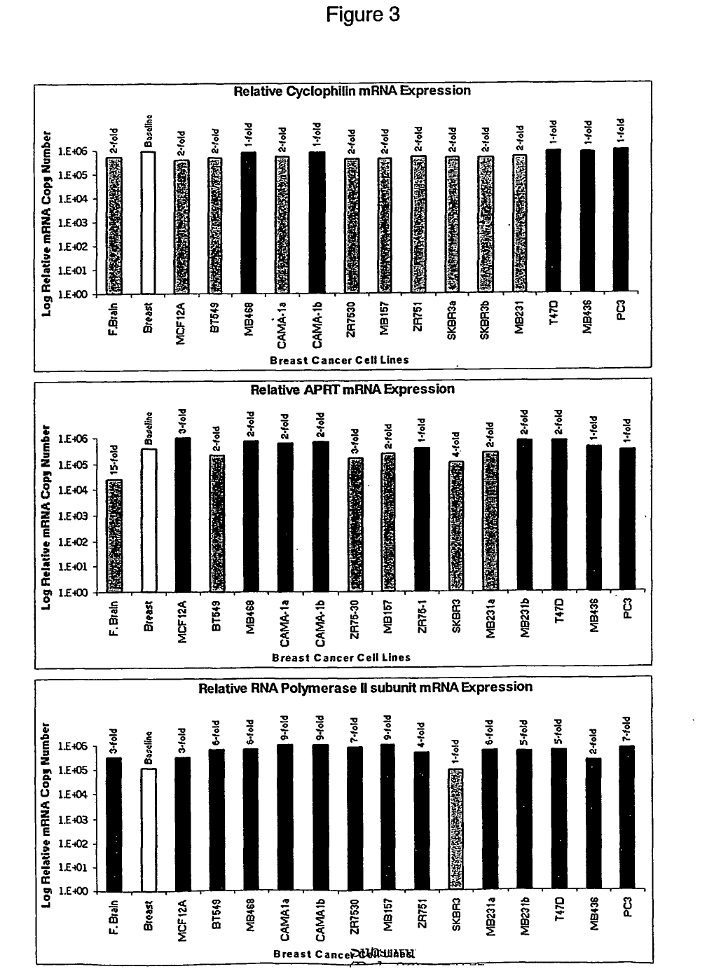

To account for variation in sample-to-sample starting template concentrations, RiboGreen™ RNA quantitation (Molecular Probes) was used to accurately assay 1 μg of normal tissue and breast cancer cell line RNA for cDNA synthesis. Selected housekeeping gene expression levels were then analyzed in all samples to determine the most accurate endogenous control for data normalization. Housekeeping amplicons included Esterase D (Accession. Number M13450), Cyclophilin (Accession Number X52851), APRT (Accession Number M16446) and RNA Polymerase II (Accession Number Z47727). Primer sequences used for RT-PCR analysis are listed in Table 2. As Cyclophilin displayed the least variable expression profile (FIG. 3), calculated MTG16 copy numbers were divided by the respective Cyclophilin amplicon copy number for each breast cancer cell line and normal tissue analyzed. MTG16 copy numbers in normalized normal breast cDNA were arbitrarily set to a ‘baseline’ of 1.0e+06 copies. Breast cancer cell lines and other normal tissue cDNA copy numbers were calculated relative to the ‘baseline’. Data is expressed as log relative mRNA copy number. Note: replicate cell lines (a and b) represent independent cell cultures, total RNA isolation and reverse transcription reactions. Replicates served as another level of control to monitor the variability in gene expression resulting from differences in cell confluency, total RNA integrity and reverse transcription efficiencies. FIGS. 3-6 show the results from these experiments.

FIG. 3 provides a summary of the degree of variation seen in mRNA expression levels between cDNA samples for three of the house-keeping genes analysed, Cyclophilin, RNA polymerase II subunit and APRT. As can be seen, expression was relatively uniform between the normal tissues and cancer cell lines. Three-way combinations for normalization between Cyclophilin, RNA polymerase II subunit and APRT demonstrated a mean 7-fold and maximum 50-fold variance in mRNA expression level between samples. The significance of variable mRNA expression levels within a gene of interest may therefore reasonably be evaluated based on these normalization results. A predicted aberrant decrease in gene of interest mRNA copy number of ˜100 fold in breast cancer cell lines relative to a ‘baseline’ normal breast expression level was therefore considered to be significantly abnormal.

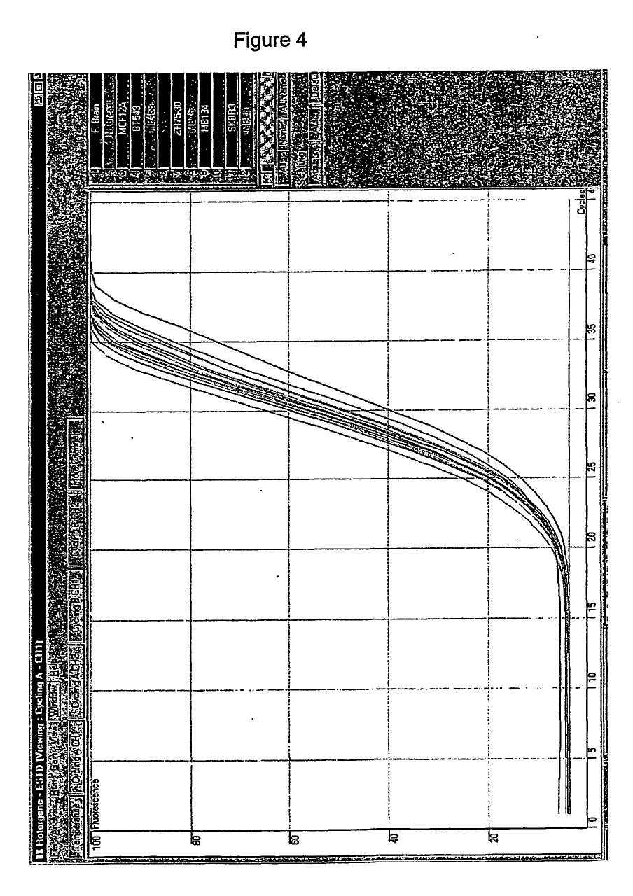

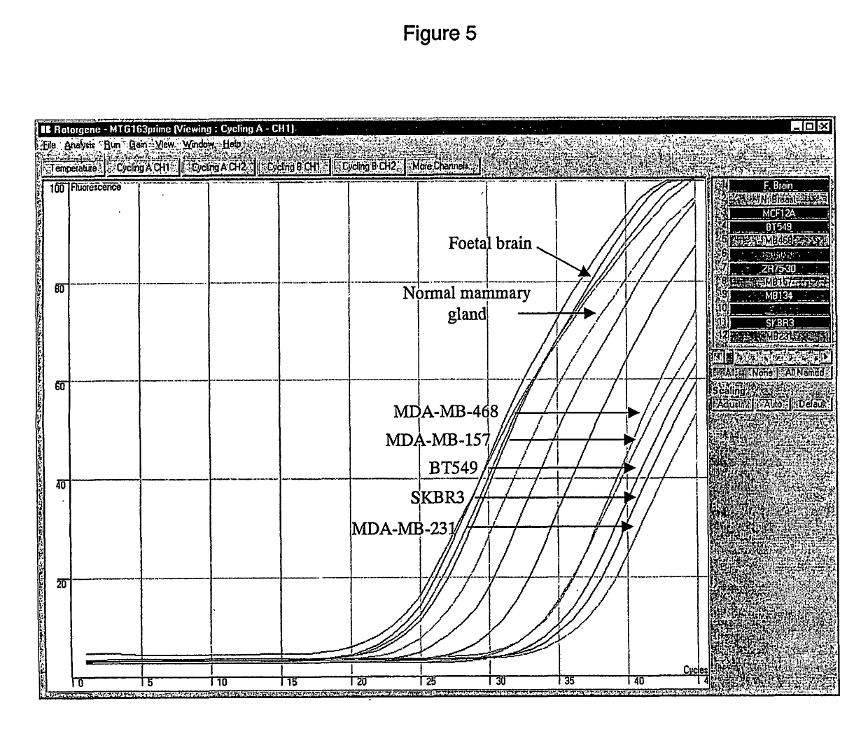

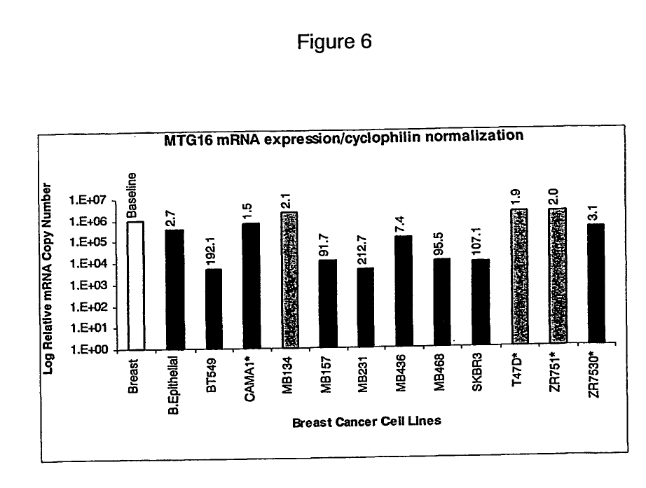

FIG. 4 provides an example of the RotorGene 2000 output for cDNA templates amplified with Esterase D specific primers. As can be seen from this figure, successful normalisation of each cDNA template was achieved. FIG. 5 shows the RotorGene 2000 output for cDNA templates amplified with MTG16 specific primers. Decreased expression of the MTG16 gene was seen in the breast cancer cell lines MDA-MB-468, MDA-MB-157, BT549, SKBR3 and MDA-MB-231 and corresponded exactly to those identified as being decreased in expression in the semi-quantitative analysis shown in FIG. 2. FIG. 6 provides a summary of the degree of variation in expression of MTG16 in a number of breast cancer cell lines compared to normal controls. A comparison between both the semi-quantitative and quantitative RT-PCR results for MTG16 expression shows consistent and significant down-regulation of the expression of the MTG16 gene in a number of breast cancer cell lines.

This aberrant loss of gene expression may result from mechanisms such as mutation or promoter methylation.

Other methods to detect MTG16 expression levels may be used. These include the generation of polyclonal or monoclonal antibodies, which are able to detect relative amounts of both normal and mutant forms of MTG16 using various immunoassays such as ELISA assays (See Example 10 and 11).

### EXAMPLE 8

### Analysis of Tumours and Cell Lines for MTG16 Mutations

The MTG16 gene was screened by SSCP analysis in DNA isolated from tumours from series 1 as well as a subset of series 2 tumours (not shown in FIG. 1) that displayed loss of the whole long arm of chromosome 16. In total 55 primary breast tumours with 16q LOH were examined for mutations.

A number of cell lines were also screened for mutations. These included 22 breast cancer cell lines (Hs578T, BT549, MB468, CAMA-1, ZR75-30, MB157, MB134, ZR75-1, SKBR3, MB231, T47D, MB436, BT483, MCF7, BT20, MB175, BT474, DU4475, MB361, MB415, MB453 and UACC893), 2 prostate cancer cell lines (LNCAP and PC3) and 3 normal breast epithelial cell lines (MCF12A, HBL100 and Hs578Bst). All cell lines were purchased from ATCC, grown according to manufacturers conditions, and DNA isolated from cultured cells using standard protocols (Wyman and White, 1980; Sambrook et al., 1989).

MTG16 exons were amplified by PCR using flanking intronic primers, which were labeled at their 5′ ends with HEX. An exception was made for exon 12 due to its size, such that it was split into 2 overlapping amplicons. Table 2 lists the sequences of all primers used for the SSCP analysis and the expected amplimer sizes. Primer sequences are represented by the SEQ ID Numbers: 17-42.

Typical PCR reactions were performed in 96-well plates in a volume of 10 ul using 30 ng of template DNA. Cycling conditions were an initial denaturation step at 94° C. for 3 minutes followed by 35 cycles of 94° C. for 30 seconds, 60° C. for 90 seconds and 72° C. for 90 seconds. A final extension step of 72° C. for 10 minutes followed. Twenty ul of loading dye comprising 50% (v/v) formamide, 12.5 mM EDTA and 0.02% (w/v) bromophenol blue were added to completed reactions which were subsequently run on 4% polyacrylamide gels and analysed on the GelScan 2000 system (Corbett Research, AUS) according to manufacturers specifications.

Tables 3-5 show the results from the mutation analysis of the MTG16 gene. An intronic polymorphism was detected in the exon 5 amplicon and was common to a number of samples. An intronic polymorphism in the exon 10 amplicon was also found, however it was only seen in two breast cancer cell lines. Coding sequence polymorphisms were also identified, however the base change was seen in both the tumour and corresponding normal constitutional DNA in each instance. A total of five tumour samples had a polymorphism in exon 2, which gave no amino acid change (c699G→A in MTG16a or c-16 G→A in MTG16b and c752G→A in MTG16a or c38G→A in MTG16b), while a polymorphism in exon 4 of the ZR75-30 cell line again led to no amino acid change (c954A→G in MTG16a or c165A→G in MTG16b). Finally, breast cancer cell line MDA-MB-175 had a nucleotide substitution in exon 2 (c763C→A in MTG16a or c49C→A in MTG16b) which gave rise to a proline to threonine amino acid change (P255T in MTG16a or P17T in MTG16b). These amino acids are similar in structure and the significance of this change is not known at this stage.

### EXAMPLE 9

### Functional Analysis of the MTG16 Gene

MTG16 Expression in Primary Tumours

To explore further the down-regulation of expression of MTG16 in breast cancer, RNA in situ hybridization was used to examine the levels of MTG16 expression in primary breast tumours.

Before tissue mounting, previously cleaned glass slides (76 mm×26 mm) were acid washed in Chromic acid for 10 minutes, rinsed thoroughly in distilled water, soaked in silane solution (2% v/v 3-aminopropyltriethoxysilane in acetone) (APES) for 1 minute then washed three times in distilled water for 1 minute each before being left to dry overnight at room temperature. Formalin fixed, paraffin embedded archival tissue sections, cut at a 4 μm thickness, were mounted on APES treated slides and baked for 2 hours at 65° C. Sections were dewaxed in xylene followed by rehydration in 100%, 90%, 70% alcohol and DEPC-treated water.

For probe preparation, a 483 bp digoxigenin-labelled antisense RNA probe was generated from the 3′ untranslated region of the MTG16 gene using the primers 5′GACAGCAGAGCAGATGCCG3′ (SEQ ID NO: 43) and 5′ GCAAGGTAGTTCACAAGTATG 3′ (SEQ ID NO: 44). This product was sub-cloned into the pGEM-t vector (Promega) using manufacturers recommendations. Digoxigenin labelled probes were subsequently generated from this construct by in vitro transcription using the DIG RNA labelling kit (SP6/T7)(Roche). The same RNA probe in a sense orientation was also generated and used as a negative control. In addition, 202 bp antisense and sense beta-actin probes were generated and used to confirm RNA integrity. Primer sequences used for beta-actin probe preparation were 5′GGCGGCACCACCATGTACCCT3′ (SEQ ID NO: 45) and 5′AGGGGCCGGACTCGTCATACT3′ (SEQ ID NO: 46)(Strassburg et al., 1997). To estimate probe concentrations, serial dilutions of labeled probes and RNA concentration standards were spotted onto a nylon membrane and hybridized with a 1:5000 dilution of sheep anti-digoxigenin F_{ab} fragments covalently coupled to alkaline phosphatase. Addition of the chromogenic substrates NBT and BCIP enabled subsequent immunodetection of the relative concentration of each RNA probe based on a comparison to the concentration standards.

Prior to hybridisation sections were pretreated with PBS (140 mM NaCl, 2.7 mM KCl, 10 mM Na₂HPO₄, 1.8 mM KH₂PO₄) for 5 minutes, treated twice with PBS/100 mM glycine for 5 minutes followed by a PBS/0.3% V/v Triton X-100 treatment for 15 minutes. Subsequently, sections were washed twice with PBS, and were then permeablised by microwave treatment. This involved bringing sections to boil in citrate buffer (10 mM tri-sodium citrate pH 6.0) by microwaving (1000 W) followed by a 10 minute cooling step on low heat. Following this, sections were washed twice in PBS for 5 minutes, twice in TEA buffer contain acetic anhydride (0.1 M triethanolamine, pH 8.0, 0.25% v/v acetic anhydride) and incubated with prehybridisation buffer consisting of 4×SSC (150 mM NaCl, 15 mM sodium citrate, pH 7.2) and 50% v/v deionised formamide) in a humid chamber for 10 minutes at 37° C.

Mounted tissue sections were drained and 30 ul of hybridization buffer (40% Deionised formamide, 10% dextran sulfate, 1× Denhardt's solution, 4×SSC, 10 mM DTT, 1 mg/ml yeast t-RNA, 1 mg/ml denatured sheared herring sperm DNA) was added. Approximately 10 ng of the appropriate DIG-labelled RNA probes was denatured at 80° C. for 10 minutes and added to the hybridization solution. This solution was overlaid with plastic coverslips then incubated at 52° C. overnight in a humid chamber. Next day, coverslips were removed by immersing slides in 2×SCC and unbound probe was removed by washing in a shaking water bath with the following washing regimen: 2×SCC, 2×15 minutes, 42° C.; 1×SCC, 2×15 minutes, 42° C.; 0.1 SSC, 2×30 minutes, 42° C. Tissues sections were then washed twice in buffer 1 (100 mM Tris-HCl, pH 7.5, 150 mM NaCl) for 10 minutes, blocked for 30 minutes with a solution of buffer 1 containing 0.1% Triton X-100 and 2% normal sheep serum at room temperature, then incubated for 2 hours in a humid chamber with buffer 1 containing 0.1% Triton X-100, 1% normal sheep serum and 1:500 dilution of sheep anti-Digoxigenin F_{ab} fragments covalently coupled to alkaline phosphatase (Roche). Following this, sections were washed twice in buffer 1 for 10 minutes and once in buffer 2 (100 mM Tris-HCl, pH 9.5, 100 mM NaCl, 50 mM MgCl₂) for 10 minutes which contained the chromogenic substrates NBT/BCIP and levamisole (1 mM). Colour reactions were allowed to proceed up to 24 hours and reactions were stopped with buffer 3 (10 mM Tris-HCl pH 8.1, 1 mM EDTA). The slides were rinsed in distilled water and counter stained in a 0.1% solution of methyl green, rinsed and mounted in glycerol:PBS (9:1).

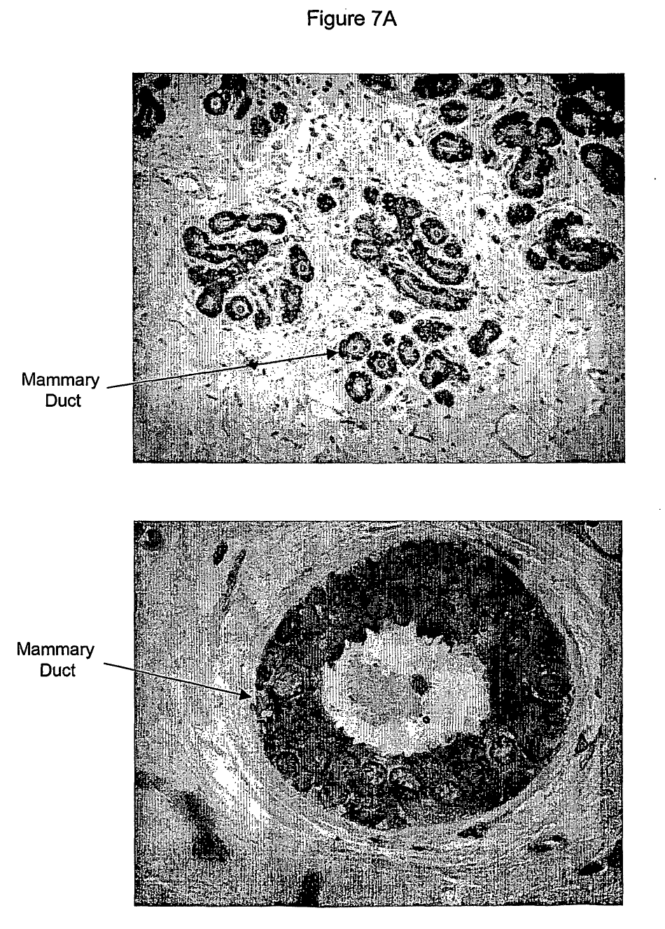

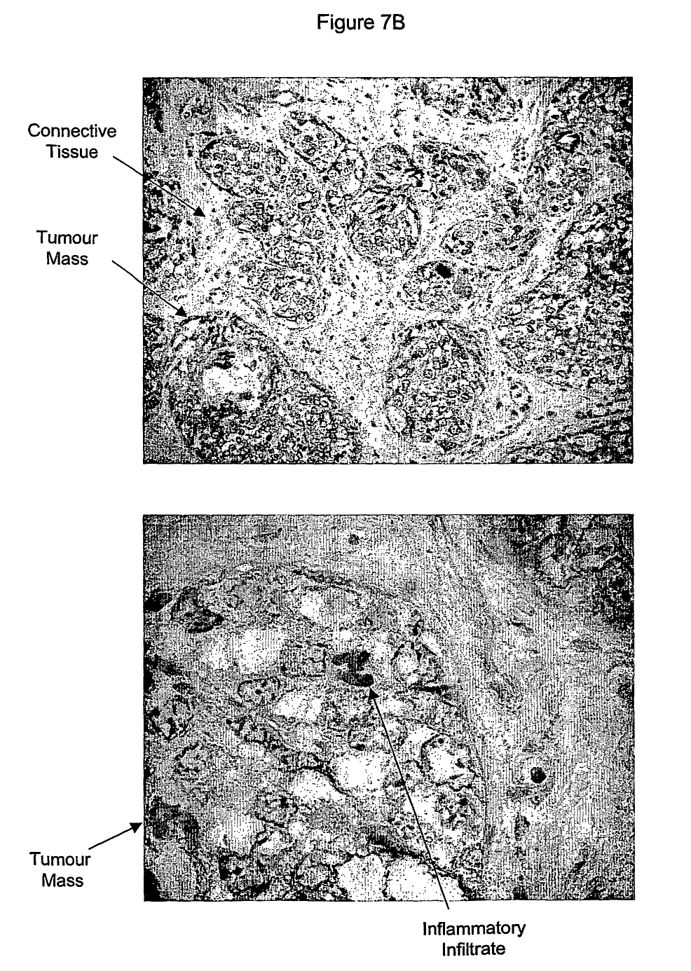

From these experiments, strong expression of the MTG16 gene was seen in each of the three normal mammary gland tissue section specimens analysed. In contrast, significant and specific reduction or complete loss of MTG16 RNA expression was found in 12 out of 22 primary breast tumour tissue sections studied. Of these 22 tumours, 5 had restricted LOH on 16q24 with 4 of these showing weak or negative MTG16 mRNA staining. FIG. 7 provides an example of MTG16 expression analysis from breast tumour tissue sections prepared from the same tissue block. Normal breast epithelial cells present in the tumour block show strong expression of MTG16 mRNA (FIG. 7A) while analysis of tumour cells shows poor or no expression of MTG16 mRNA (FIG. 7B).

This data confirms that the MTG16 gene is significantly down-regulated in its expression in breast cancer samples confirming its role as a tumour suppressor in the 16q24.3 LOH region identified by our studies.

Suppression of Human Breast Cancer Cell Growth by MTG16

The effect on cell growth, through re-introduction of MTG16 protein into breast cancer cell lines, was examined. Four different breast cancer cell lines were chosen with three of these (SK-BR-3, MDA-MB-231 and MDA-MB-468) showing likely LOH at 16q24.3 and reduced expression of MTG16 through RT-PCR studies. The final cell line (MCF7) in contrast did not show reduced MTG16 expression and did not show likely 16q24.3 LOH.

Initially a full length MTG16 (MTG16b isoform) cDNA was cloned into the retroviral expression vector pLNCX2 (Clontech). MTG16 was amplified from fetal spleen total RNA using a Myc-tag containing forward primer 5′ ATGGAGCAG AAGCTGATCAGCGAGGAGGACCTGATGCCGGACTCCCCAGCGGA 3′ (SEQ ID NO: 47) and reverse primer 5′ TCAGCGGGGCACGGTGTCCA 3′ (SEQ ID NO: 48). The resultant amplicon was subcloned into the SalI/ClaI sites of the pLNCX2 vector using standard methods (Sambrook et al., 1989).

The chosen breast cancer cell lines were subsequently infected with VSV-G pseudo-typed retroviruses expressing Myc-tagged MTG16 together and a Neomycin selectable marker. This first involved plating HEK 293T packaging cells on 10 cm tissue culture dishes at 40% confluence in Dulbecco's modified Eagle's medium (DMEM, Gibco) supplemented with 10% calf serum, 2 mM L-glutamine and 10 mg/L of penicillin and gentamicin. The cells were incubated for 24 hours at 37° C. and 5% CO₂. Following this, the HEK 293T cells were transfected with 10 ug of pLNCX2 retroviral vector constructs, 8 ug of pVPack-VSV-G (Stratagene), 8 ug of pVPack-GP (Stratagene) and 60 ul of Lipofectamine 2000 reagent (Gibco BRL) according to manufacturers specifications. Cells were grown in OptiMEM (Gibgo BRL) without fetal calf serum and antibiotics and following a 16 hour incubation at 37° C. and 5% CO₂, the medium was replaced and grown a further 32 hours. Viral containing supernatants were then harvested and filtered through 0.45 um Minisart syringe filters (Sartorius AG, Germany) and polybren was added to a final concentration of 8 ug/ml. The selected breast cancer cell lines were plated in 6-well plates at 60% confluency and were infected with the purified virus supernatants. Cells were incubated for 2 days at 37° C. and 5% CO₂.

To study the effect of MTG16 on monolayer colony formation 5×10³ infected tumour cells were plated in 6-well plates and a colony formation assay was performed in 500 ng/ml of G418. After two weeks of selection cells were fixed in 3.7% formaldehyde in PBS, stained with Giemsa (Sigma) and dried for subsequent quantification. Colonies visible in each well without magnification were counted and average values were determined for each recombinant retrovirus (mean+SEM).

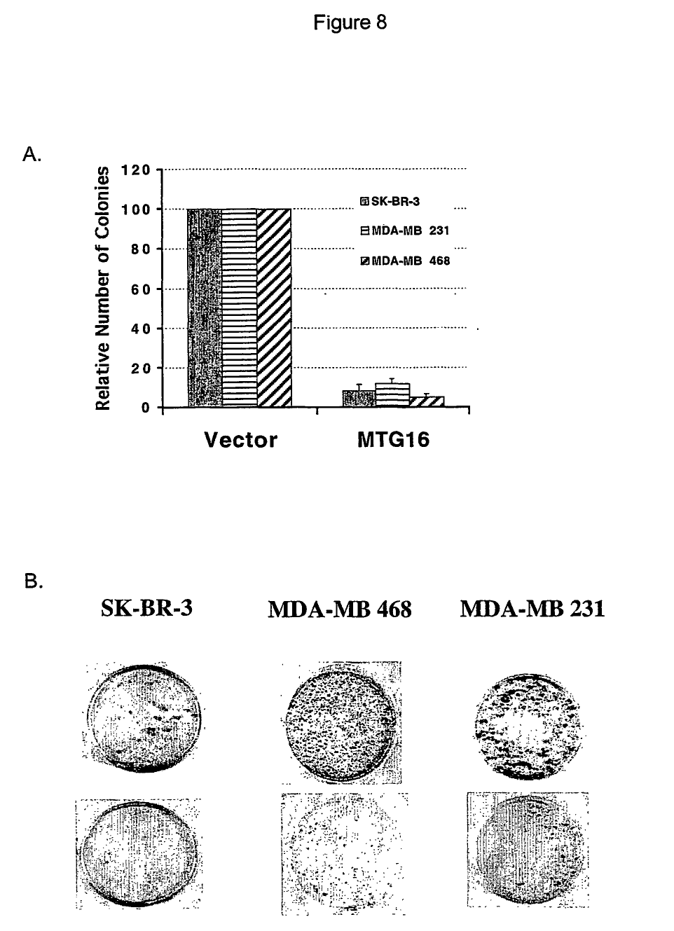

Results of these studies showed that the expression of MTG16 in the SK-BR-3, MDA-MB-231 and MDA-MB-468 breast cancer cell lines dramatically reduced colony growth (up to 25 fold) compared to Neomycin only expressing controls (FIG. 8). However the effect of MTG16 retroviral expression in MCF-7 breast cancer cells was not as pronounced as only an approximately 25% reduction in colony numbers under the same experimental conditions was observed.

To rule out the possibility that the observed low number of surviving colonies from MTG16 expressing breast cancer cells was due to a low retroviral infection efficiency, infected cell lines were stained with anti-Myc monoclonal antibodies to visualise MTG16 transduced cells. In all cell lines, at least 50-70% of the infected cells expressed Myc-tagged MTG16 protein.

It is interesting to note that attempts to expand surviving colonies into cell lines stably producing MTG16 have failed with both SK-BR-3 and MDA-MB-468 cells. MDA-MB-231 selected clones did survive expansion, however they were rapidly losing MTG16 even after short (2 weeks) culturing in selectable media. This observation possibly reiterates the fact that the effect of MTG16 expression is detrimental to cancer cell growth.

The effect of MTG16 on the ability of MDA-MB-231 and MDA-MB-468 breast cancer cell lines to form colonies in an anchor-independent manner was also examined. SK-BR-3 was omitted as this cell line is non-tumorigenic and does not form defined colonies in semi-solid media (Thompson et al, 1992). Cells infected with specific (MTG16 expressing) or control (Neo only) retroviral particles were suspended in soft agar containing G418 and colony numbers were scored after two to three weeks of incubation. Data collected from these assays paralleled those obtained on plastic surface with MTG16 strongly and specifically inhibiting colony formation in the chosen breast cancer cell lines.

MTG16 Localisation

To gain insight into the physiological function of the MTG16 protein, the intracellular localization of the MTG16 protein was examined. An MTG16-GFP fusion protein was generated using the primers 5′ ATGCCGGACTCCCCAGCGGA 3′ (SEQ ID NO: 49) and 5′ TCAGCGGGGCACGGTGTCCA 3′ (SEQ ID NO: 48) and expressed in the MDA-MB-468 cell line. Transfected cells were cultivated on glass coverslips and fixed for 15 minutes at room temperature in PBS containing 3.7% formaldehyde. Cells were then rinsed 3 times with PBS and finally permeabilised for 5 minutes at 4° C. in PBS containing 0.4% Triton X-100. Cells were then incubated with a 1:500 dilution of a monoclonal Myc antibody (Santa Cruz) for 1 hour at room temperature followed by a 1 hour incubation with a 1:600 dilution of an FITC-conjugated sheep anti-mouse IgG (Silenus, Australia). Coverslips were mounted with Vectashield mounting liquid containing DAPI for DNA staining and cells were visualised using fluorescence microscopy.

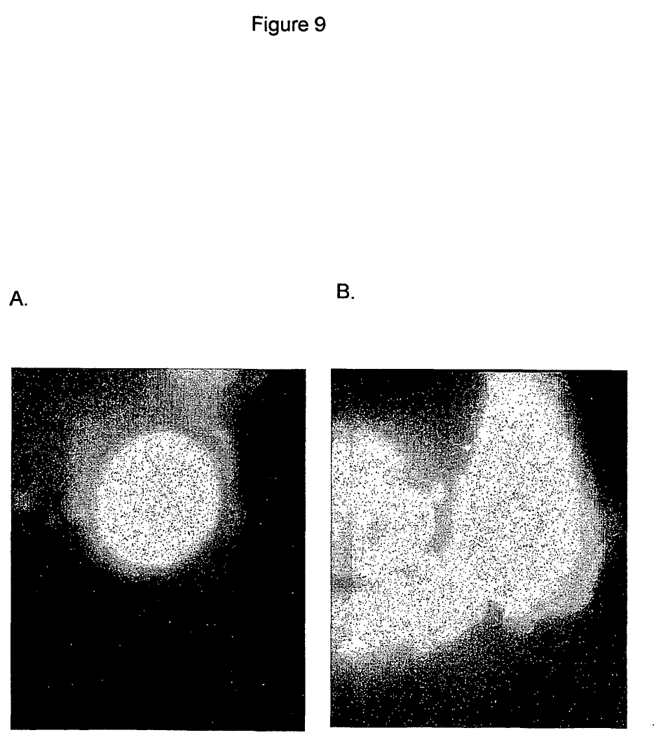

GFP-tagged MTG16 was found to produce a distinct punctate pattern over weaker diffuse staining in the cell nuclei (FIG. 9A) compared to even cytoplasmic and nuclear distribution of the GFP alone (FIG. 9B). To establish whether the large GFP molecule could interfere with the localisation of the tagged protein the localisation of the Myc-tagged MTG16 in the same cells fixed and stained with anti-Myc monoclonal antibodies was examined. Myc-tagged protein showed the same pattern of nuclear localisation.

Having established MTG16 protein nuclear localisation we next addressed the possibility of this protein being a transcriptional regulator, since other members of the ETO family of proteins have been implicated in transcriptional repression. As the MTG16 protein does not contain a conserved DNA binding domain, in order to study its transcriptional regulatory properties the full length MTG16 was fused to the DNA binding domain of the yeast GAL4 transcription factor present in the pM expression vector (Clontech) to generate the pMMTG16 construct. To generate control constructs, the KRAB repression domain of the mouse NK10 protein (amino acids 1 to 112) (Thiel et al., 2000) was fused to the GAL4 DNA binding domain of vector pM to generate the pMNK10 positive control. The KRAB domain was amplified from NIH3T3 cell total RNA using primers 5′ TATCGAATTCCCAGCACACAC 3′ and 5′ TATCGGATCCTCACCTGGTC 3′. This positive control construct had been previously well characterised under the same experimental conditions (Thiel et al., 2000). As a negative control, five copies of the GAL4 DNA binding sites were introduced directly upstream of the HSV1 thymidine kinase promoter to create the CAT gene reporter construct GAL4CAT2.

A total of 1×10⁵ 293T cells were transfected in 6-well plates with 1 μg of reporter construct, up to 3 μg of specific and control GAL4 fusion expression vectors and 500 ng of β-gal expression plasmid Lipofectamine 2000 reagent. Twenty four hours post transfection, cells were lysed and CAT concentration was estimated using the CAT ELISA kit (Roche) according to manufacturers specifications. The β-Galactosidase assay (Stratagene) was performed as an internal control of transfection efficiency and CAT values were then normalised with respect to β-galactosidase concentration.

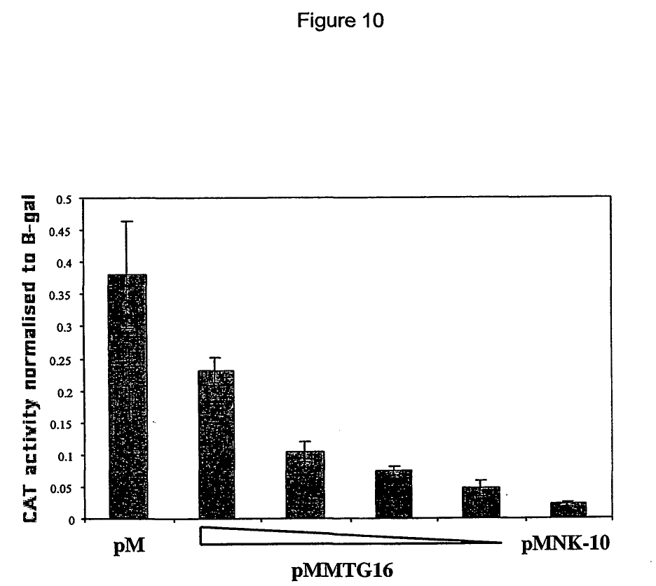

Results from this assay show that MTG16 can act as a strong transcriptional repressor (FIG. 10). Activity from the CAT reporter was reduced up to 10 fold in a specific and dose-dependent manner when pMMTG16 was contransfected with the GAL4CAT2 reporter construct in 293T cells. In a separate experiment using the NIH-3T3 cell line, it was shown that MTG16 transcriptional repression activity is not cell type specific.

CpG Island Methylation as Down-Regulator of MTG16 Expression

Loss of heterozygosity of alleles by mechanisms such as deletion, uniparental disomy or somatic recombination concomitant with mutation in retained alleles can result in complete loss of tumour suppressor gene function. Transcriptional silencing by CpG promoter hypermethylation functions as an allele specific epigenetic alternative to mutation. Evidence to support this hypothesis substantiates primarily from the findings that methylation silencing of tumour suppressor genes, such as APC, MLH1, p16^{INK4a}, pRb, pVHL, and p19^{ARF}, can occur in conjunction with LOH and/or mutation, hence defining methylation as one potential ‘knockout’ in biallelic inactivation.

CpG methylation regulates gene expression by remodelling chromatin structure to prevent binding and assembly of transcription factors to promoter elements hence repressing transcription. Remodelling is via either major-groove clashes of methylated promoter sequence with transcription factors or, more generally, via a time-dependent “closing” in chromatin structure into a condensed state.

MTG16 exon 1a and exon 1b 5′-UTR variants suggests two independent promoters may drive transcription. Such alternative promoters may dictate transcriptional kinetics and modes of induction specific to the MTG16a or MTG16b isoforms. Preliminary real-time studies differentiating between MTG16a and MTG16b expression levels indicate that the b isoform is the predominately down-regulated transcript variant in breast cancer cell lines.

In silico analysis has identified a dense region of CpG dinucleotides within and adjacent to the genomic DNA sequence of MTG16 exon 1b. To determine if a correlation exists between the down-regulation in MTG16 gene expression and the methylation status of the exon 1b CpG island in breast cancer cell lines a sodium bisulfite methylation-specific PCR assay was performed. Sodium bisulfite is able to convert cytosine residues to thymidine only when the cytosine residue is unmethylated. Therefore methylated cytosine residues that are part of CpG islands will remain untouched by this chemical.

To perform this assay, breast cancer cell line DNA was first isolated. Breast cancer cell lines including those showing consistent down-regulation in the expression of MTG16 from quantitative RT-PCR experiments were chosen. Cells were grown as described above and DNA was isolated using the Trizol reagent (Gibco BRL).

Breast cancer cell line DNA was diluted 2 μg in 50 μl water, treated with 5.5 μl of 2 M NaOH, incubated at 37° C. for 15 minutes, 93° C. for 2 minutes then chilled. Denatured DNA was mixed with 30 μl of 10 mM hydroquinone (Sigma), 520 μl 3 M NaHSO₃ (Sigma), overlaid with paraffin oil and incubated in the dark for 16 hours at 55° C. Paraffin oil was removed and DNA recovered with DNA Wizard Cleanup (Promega). DNA, resuspended in 50 μl of water, was treated with 5.5 μl of 3 M NaOH, incubated at 37° C. for 15 minutes and neutralized with 17 μl of 10 mM ammonium acetate (pH 7.0). DNA was precipitated in 2.5 volumes of cold 100% ethanol and 1/10^{th} glycogen, washed with 70% ethanol, resuspended in 20 μl water and stored at −80° C.

Amplification of wild-type, unmethylated and methylated MTG16 alleles were performed by real-time PCR using a final 25 μl reaction mix, SYBR Green detection and amplification cycling as described above. PCR template consisted of 50 ng NaHSO₃ modified breast cancer cell line DNA. Primers were designed specific to the CpG island spanning MTG16 exon 1b and adjacent 5′ genomic sequence. The primer sequences used are shown in Table 2 and are represented by the SEQ ID Numbers: 50-55. Real-time products were visualized with ethidium bromide on 2.5% agarose gel electrophoresis prior to real-time quantification as described above. Wild-type, unmethylated and methylated products were purified by QIAquick gel extraction (Qiagen) and sequenced with ABI Prism Big-Dye™ Terminator (PE Biosystems).

Amplification of methylated 1b alleles was detected in MDA-MB-231 and MDA-MB-468, two breast cancer cell lines that showed significant down-regulation of MTG16 expression. Sequence analysis revealed 100% methylation of 41 CG dinucleotides within 250 bp of exon 1b and adjacent 5′ sequence in these cell lines.

To examine the effect of methylation on MTG16 expression in MDA-MB-231 cells further, the cell line was grown to 80% confluency and resuspended at 1.0×10⁵ cells/ml in 10 ml RPMI+FCS or OPTI-MEM per 90 mm petri-dish. Cells were then incubated for 156 hours with 5.0 μm 5-aza-2′-deoxycytidine (5-AzaC), a chemical that demethylates DNA. Treated cells were replenished with fresh media solution and 5-AzaC every 12 hours for the duration of the experiment. DNA was then isolated using the TRIzol Reagent (Gibco BRL) and real-time re-expression and methylation-specific PCR analysis was repeated as described above.

Treatment of the breast cancer cell line MDA-MB-231 with 5-AzaC resulted in marked demethylation and re-expression of MTG16 alleles as detected by real-time PCR. These results indicate that the potential for LOH of MTG16 alleles concomitant with methylation silencing of retained alleles, an alternative mechanism to mutation, may lead to complete or abnormal loss of MTG16 function in breast cancer.

Protein Interaction Studies

The ability of MTG16 protein to bind known and unknown protein can be examined. Procedures such as the yeast two-hybrid system are used to discover and identify any functional partners. The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

The nature of the MTG16 interacting genes and proteins can also be studied such that these partners can also be targets for therapeutic and diagnostic development.

Structural Studies

MTG16 recombinant proteins can be produced in bacterial, yeast, insect and/or mammalian cells and used in crystallographical and NMR studies. Together with molecular modeling of the protein, structure-driven drug design can be facilitated.

### EXAMPLE 10

### Generation of Polyclonal Antibodies Against MTG16

The knowledge of the nucleotide and amino acid sequence of MTG16 allows for the production of antibodies, which selectively bind to MTG16 protein or fragments thereof. Antibodies can also be made to selectively bind and distinguish mutant from normal protein. Antibodies specific for mutagenised epitopes are especially useful in cell culture assays to screen for malignant cells at different stages of malignant development. These antibodies may also be used to screen malignant cells, which have been treated with pharmaceutical agents to evaluate the therapeutic potential of the agent.

To prepare polyclonal antibodies, short peptides can be designed homologous to the MTG16 amino acid sequence. Such peptides are typically 10 to 15 amino acids in length. These peptides should be designed in regions of least homology to the mouse orthologue to avoid cross species interactions in further down-stream experiments such as monoclonal antibody production. Synthetic peptides can then be conjugated to biotin (Sulfo-NHS-LC Biotin) using standard protocols supplied with commercially available kits such as the PIERCE™ kit (PIERCE). Biotinylated peptides are subsequently complexed with avidin in solution and for each peptide complex, 2 rabbits are immunized with 4 doses of antigen (200 μg per dose) in intervals of three weeks between doses. The initial dose is mixed with Freund's complete adjuvant while subsequent doses are combined with Freund's Immuno-adjuvant. After completion of the immunization, rabbits are test bled and reactivity of sera assayed by dot blot with serial dilutions of the original peptides. If rabbits show significant reactivity compared with pre-immune sera, they are then sacrificed and the blood collected such that immune sera can be separated for further experiments.

### EXAMPLE 11

### Generation of Monoclonal Antibodies Specific for MTG16

Monoclonal antibodies can be prepared for MTG16 in the following manner. Immunogen comprising intact MTG16 protein or MTG16 peptides (wild type or mutant) is injected in Freund's adjuvant into mice with each mouse receiving four injections of 10 to 100 ug of immunogen. After the fourth injection blood samples taken from the mice are examined for the presence of antibody to the immunogen. Immune mice are sacrificed, their spleens removed and single cell suspensions are prepared (Harlow and Lane, 1988). The spleen cells serve as a source of lymphocytes, which are then fused with a permanently growing myeloma partner cell (Kohler and Milstein, 1975). Cells are plated at a density of 2×10⁵ cells/well in 96 well plates and individual wells are examined for growth. These wells are then tested for the presence of MTG16 specific antibodies by ELISA or RIA using wild type or mutant MTG16 target protein. Cells in positive wells are expanded and subcloned to establish and confirm monoclonality. Clones with the desired specificity are expanded and grown as ascites in mice followed by purification using affinity chromatography using Protein A Sepharose, ion-exchange chromatography or variations and combinations of these techniques.

### Industrial Applicability

The MTG16 gene has been shown to be a tumour suppressor gene implicated not only in breast cancer, but in the tumourigenic process in general. The MTG16 gene therefore is useful in methods for the early detection of cancer susceptible individuals as well as in diagnostic, prognostic and therapeutic procedures associated with these disease states.

TABLE 1
Splice Sites of the MTG16 Gene

| | | | | | | |
|---|---|---|---|---|---|---|
| | Size | 3′ Splice site | Consensus strength | 5′ Splice site | Consensus strength | Intron size |
| Exon | (bp) | (intron/exon) | (%) | (exon/intron) | (%) | (bp) |

| | | | | | | |
|---|---|---|---|---|---|---|
| 1a | | 5′UTR | | GGCGGCCCAG/gtaagaagct | 94.34 | >80427 |
| 1b | | 5′UTR | | CCCCCCGACC/gtaagtgccg | 72.1 | >39000 |
| 2 | 153 | ttggtcgcag/CCCCAGTGGA | 81.87 | CCACACACAC/gtaagtagcc | 86.5 | 3354 |
| 3 | 75 | ttgcttacag/ATCGAGAGGA | 96.0 | GTCTGTCTCT/gtaagtaaaa | 72.77 | 5594 |
| 4 | 242 | cctcctgcag/TGATGAACGG | 87.03 | GGGCCTGGTG/gtgagttggg | 81.84 | 235 |
| 5 | 90 | tcccctgcag/AACTCGACAT | 93.5 | CTTCCTGAAG/gtaatgcgaa | 74.62 | >4713 |
| 6 | 182 | caccccacag/GCAAACCTGC | 89.68 | CGCCCGACAG/gtacctgtgg | 68.4 | 692 |
| 7 | 224 | ttggggctag/GACCAAAGAG | 70.41 | CGGCCGCTTG/gtgagcagcc | 76.4 | 2285 |
| 8 | 86 | ccatccacag/TGGTGCCTGG | 82.34 | CCTCAACAAC/gtgagtgtcc | 76.63 | 1187 |
| 9 | 199 | ccgcccccag/CTCCTGAACT | 80.48 | CCTCAGCTAG/gtgcgtgtgg | 78.48 | 503 |
| 10 | 69 | cctgtttcag/ACGTGCCTCG | 82.57 | AGGAAGGCTG/gtgagtgggg | 81.84 | 1260 |
| 11 | 191 | tcctttgcag/AAGAGGCCGT | 91.32 | CTCCAGCGAG/gtagggccac | 77.98 | 1995 |
| 12 | 2406 | ccccacccag/AGCTGCTGGA | 85.38 | 3′UTR | | |

TABLE 2
Oligonucleotide Primers for Analysis of MTG16

| | Nucleotide | |
|---|---|---|
| Primer | sequences | Size |
| set | (5′-3′) | (bp) |

| | | |
|---|---|---|
| SSCP¹ | | |
| exon 1a | GTCCTGGGCTCCAGGTTG | 271 |
| | GAAGCTCTAAGGAGTCACAG | |
| exon 2 | TTGCACTTAGCCTGCTTCAC | 250 |
| | GCCTCCCCTGAAACACCTG | |
| exon 3 | AAAAATCACTCTGAGAAGTAGG | 251 |
| | TGTTGGGCCAGCTGAGGATG | |
| exon 4 | TGTGTCCTCATGTCCGCTTC | 323 |
| | CGGAGGGAATATGCATGTCC | |
| exon 5 | CTGCCTCCAACACGGAAGC | 265 |
| | TCCACGCTGCGAAGGAGTG | |
| exon 6 | GTGCACCCCTGCATGCTAC | 316 |
| | GAGGAGGTTCCCTCTCTTAC | |
| exon 7 | GTTCATCCTATGTCCACTGC | 324 |
| | CATGTGTGCTCCTGTAACAC | |
| exon 8 | AGAATAGGGCAGAGACTGGC | 166 |
| | TGGCTGTGTGTGGACACTC | |
| exon 9 | TCTGAGGTGCTGAAGGCTG | 276 |
| | AGCACCCCGTGTCTGCTC | |
| exon 10 | GTGGCCCATCCTGTGTGAC | 188 |
| | TTCAAAGCTGAGCCGGTGAG | |
| exon 11 | TGGCCACGCGTAGGAAGTC | 305 |
| | GCAGGGGATGGGTGTCAG | |
| exon 12 (set 1) | CAGACCCAGCCCTGACTG | 151 |
| | CACACGTGGTGATGCTTCTC | |
| exon 12 (set 2) | TCTGCCAGCATCGGGACTG | 269 |
| | GTTGGCACGGTGCTGTGTC | |
| RT-PCR | | |
| MTG16² | GGGTTTGTGCCCAGTTAGAA | 160 |
| | TATGAAAAGTCACAGGGGGC | |
| Esterase D^{2,3} | GGAGCTTCCCCAACTCATAAATGCC | 453 |
| | GCATGATGTCTGATGTGGTCAGTAA | |
| Cyclophilin³ | GGCAAATGCTGGACCCAACAAA | 355 |
| | CTAGGCATGGGAGGGAACAAGGGAA | |
| APRT ³ | GACTGGGCTGCGTGCTCATCC | 316 |
| | AGGCCCTGTGGTCACTCATACTGC | |
| RNA Pol. II³ | AGGGGCTAACAATGGACACC | 300 |
| | CCGAAGATAAGGGGGAACTACT | |
| MTG16³ | GGGCCTGGTGAACTCGACATTGAC | 80 |
| | ACGGCCGCAGAGGGAAGTTGGT | |
| Methylation | | |
| Sensitive PCR | | |
| MTG16WT | GCGGGGTGCGCGCTTTGTTCCCGCGCGG | 443 |
| | CGAGACTCCCAGCGCCCGGGCGCGTCGT | |
| MTG16Methyl | GCGGGGTCGGCGTTTTGTTTTCGCGCGG | 443 |
| | CGAAACTCCCAACGCCCGAACGCGTCGT | |
| MTG16Unmethyl | TGTGTGTTTTGTTTTTGTGTGG | 459 |
| | ACTCCCAACACCCAAACACATCAT | |

Note:
¹Primers for SSCP analysis were labelled at their 5′ends with HEX.
²Primer sets used for semi-quantitative RT-PCR.
³Primer sets used for quantitative RT-PCR.

TABLE 3
SSCP Analysis of MTG16 in Microdissected Tumour/Normal Paired Breast Cancer Samples

| | | Exon | | | | | | | | | Exon | Exon | Exon | Exon |
|---|---|---|---|---|---|---|---|---|---|---|---|---|---|---|
| SAMPLES | | 1a | Exon 2 | Exon 3 | Exon 4 | Exon 5 | Exon 6 | Exon 7 | Exon 8 | Exon 9 | 10 | 11 | 12a | 12b |
| 1. | 8/96 | + | + | + | + | P(a) | + | + | + | + | + | + | + | + |
| 2. | 12/96 | + | + | + | + | + | + | + | + | + | + | + | + | + |
| 3. | 19/96 | + | + | + | + | P(a) | + | + | + | + | + | + | + | + |
| 4. | 22/96 | + | + | + | + | P(b) | + | + | + | + | + | + | + | + |
| 5. | 29/96 | + | + | + | + | + | + | + | + | + | + | + | + | + |
| 6. | 2/97 | + | + | + | + | + | + | + | + | + | + | + | + | + |
| 7. | 6/97 | + | + | + | + | + | + | + | + | + | + | + | + | + |
| 8. | 90/447 | + | + | + | + | + | + | + | + | + | + | + | + | + |
| 9. | 88/248 | + | + | + | + | + | + | + | + | + | + | + | + | + |
| 10. | 90/371 | + | + | + | + | + | + | + | + | + | + | + | + | + |
| 11. | 89/605 | + | + | + | + | + | + | + | + | + | + | + | + | + |
| 12. | 90/32 | + | + | + | + | + | + | + | + | + | + | + | + | + |
| 13. | 89/257 | + | + | + | + | + | + | + | + | + | + | + | + | + |
| 14. | 90/12 | + | + | + | + | P(a) | + | + | + | + | + | + | + | + |
| 15. | 91/587 | + | P(a) | + | + | P(a) | + | + | + | + | + | + | + | + |
| 16. | 91/250 | + | + | + | + | P(a) | + | + | + | + | + | + | + | + |
| 17. | 87/820 | + | + | + | + | + | + | + | + | + | + | + | + | + |
| 18. | 90/431 | + | + | + | + | + | + | + | + | + | + | + | + | + |
| 19. | 90/581 | + | + | + | + | + | + | + | + | + | + | + | + | + |
| 20. | 90/269 | + | + | + | + | + | + | + | + | + | + | + | + | + |
| 21. | 90/632 | + | + | + | + | P(b) | + | + | + | + | + | + | + | + |
| 22. | 88/531 | + | + | + | + | + | + | + | + | + | + | + | + | + |
| 23. | 88/38 | + | + | + | + | P(b) | + | + | + | + | + | + | + | + |
| 24. | 88/467 | + | + | + | + | + | + | + | + | + | + | + | + | + |

Note:
+: Identical to the wild-type MTG16 sequence. Exon 2: P(a) = c699G→A in MTG16a or c-16G→A in MTG16b (Lys→Lys; present in tumour and normal samples); Exon 5: Intronic G→C polymorphism (+ = G, P(a) = G/C, P(b) = C)

TABLE 4
SSCP Analysis of MTG16 in Tumour/Normal Paired Breast Cancer Samples

| | Exon | | | | | | | | | Exon | Exon | Exon | Exon |
|---|---|---|---|---|---|---|---|---|---|---|---|---|---|
| SAMPLES | 1a | Exon 2 | Exon 3 | Exon 4 | Exon 5 | Exon 6 | Exon 7 | Exon 8 | Exon 9 | 10 | 11 | 12a | 12b |
| Loss 16q24.3 | | | | | | | | | | | | | |
| 1. 204 | + | + | + | + | P(a) | + | + | + | + | + | + | + | + |
| 2. 309 | + | + | + | + | + | + | + | + | + | + | + | + | + |
| 3. 358 | + | + | + | + | + | + | + | + | + | + | + | + | + |
| 4. 367 | + | + | + | + | + | + | + | + | + | + | + | + | + |
| 5. 413 | + | + | + | + | + | + | + | + | + | + | + | + | + |
| 6. 549 | + | + | + | + | + | + | + | + | + | + | + | + | + |
| 7. 559 | + | + | + | + | + | + | + | + | + | + | + | + | + |
| 8. 589 | + | + | + | + | + | + | + | + | + | + | + | + | + |
| 9. 645 | + | + | + | + | P(a) | + | + | + | + | + | + | + | + |
| 10. 666 | + | + | + | + | P(a) | + | + | + | + | + | + | + | + |
| 11. 757 | + | + | + | + | P(a) | + | + | + | + | + | + | + | + |
| 12. 819 | + | + | + | + | + | + | + | + | + | + | + | + | + |
| 13. 919 | + | + | + | + | P(b) | + | + | + | + | + | + | + | + |
| 14. 477 | + | + | + | + | + | + | + | + | + | + | + | + | + |
| Loss 16q22.1-16q24 | | | | | | | | | | | | | |
| 15. 152 | + | + | + | + | P(a) | + | + | + | + | + | + | + | + |
| 16. 380 | + | + | + | + | + | + | + | + | + | + | + | + | + |
| 17. 670 | + | P(a) | + | + | + | + | + | + | + | + | + | + | + |
| 18. 683 | + | + | + | + | + | + | + | + | + | + | + | + | + |
| 19. 768 | + | P(a) | + | + | P(a) | + | + | + | + | + | + | + | + |
| 20. 594 | + | P(b) | + | + | + | + | + | + | + | + | + | + | + |
| Loss Whole 16q | | | | | | | | | | | | | |
| 21. 424 | + | + | + | + | P(a) | + | + | + | + | + | + | + | + |
| 22. 438 | + | P(y) | + | + | P(a) | + | + | + | + | + | + | + | + |
| 23. 439 | + | + | + | + | + | + | + | + | + | + | + | + | + |
| 24. 448 | + | + | + | + | + | + | + | + | + | + | + | + | + |
| 25. 573 | + | + | + | + | + | + | + | + | + | + | + | + | + |
| 26. 578 | + | + | + | + | P(a) | + | + | + | + | + | + | + | + |
| 27. 735 | + | + | + | + | + | + | + | + | + | + | + | + | + |
| Complex Loss | | | | | | | | | | | | | |
| 28. 355 | + | + | + | + | + | + | + | + | + | + | + | + | + |
| 29. 377 | + | + | + | + | + | + | + | + | + | + | + | + | + |
| 30. 555 | + | + | + | + | + | + | + | + | + | + | + | + | + |
| 31. 581 | + | + | + | + | P(a) | + | + | + | + | + | + | + | + |

Note:
+: Identical to wild-type MTG16 sequence. Exon 2: P(a) = c699G→A in MTG16a or c-16 G→A in MTG16b (Lys→Lys; present in tumour and normal samples); Exon 2: P(y) = c752G→A in MTG16a or c38G→A in MTG16b (Pro→Pro; present in tumour and normal samples); Exon 5: Intronic G→C polymorphism (+ = G, P(a) = G/C, P(b) = C).

TABLE 5
SSCP Analysis of MTG16 in Breast and Prostate Cancer Cell Lines

| | Exon | | | | | | | | | Exon | Exon | Exon | Exon |
|---|---|---|---|---|---|---|---|---|---|---|---|---|---|
| CELL LINES | 1a | Exon 2 | Exon 3 | Exon 4 | Exon 5 | Exon 6 | Exon 7 | Exon 8 | Exon 9 | 10 | 11 | 12a | 12b |
| 1. MCF 12A | + | + | + | + | + | + | + | + | + | + | + | + | + |
| 2. HBL 100 | + | + | + | + | + | + | + | + | + | + | + | + | + |
| 3. Hs 578 Bst | + | + | + | + | + | + | + | + | + | + | + | + | + |
| 4. Hs 578 T | + | + | + | + | + | + | + | + | + | + | + | + | + |
| 5. BT 549 | + | + | + | + | + | + | + | + | + | + | + | + | + |
| 6. MB 468 | + | + | + | + | + | + | + | + | + | + | + | + | + |
| 7. CAMA-1 | + | + | + | + | + | + | + | + | + | + | + | + | + |
| 8. ZR-75-30 | + | + | + | P(a) | P(b) | + | + | + | + | + | + | + | + |
| 9. MB 157 | + | + | + | + | + | + | + | + | + | + | + | + | + |
| 10. MB 134 | + | + | + | + | + | + | + | + | + | P(a) | + | + | + |
| 11. ZR-75-1 | + | + | + | + | + | + | + | + | + | + | + | + | + |
| 12. SK BR 3 | + | + | + | + | + | + | + | + | + | + | + | + | + |
| 13. MB 231 | + | + | + | + | + | + | + | + | + | + | + | + | + |
| 14. T47D | + | + | + | + | + | + | + | + | + | P(a) | + | + | + |
| 15. MB 436 | + | + | + | + | P(a) | + | + | + | + | + | + | + | + |
| 16. BT 483 | + | + | + | + | P(a) | + | + | + | + | + | + | + | + |
| 17. MCF 7 | + | + | + | + | + | + | + | + | + | + | + | + | + |
| 18. BT 20 | + | + | + | + | P(a) | + | + | + | + | + | + | + | + |
| 19. MB 175 | + | P(z) | + | + | P(a) | + | + | + | + | + | + | + | + |
| 20. BT 474 | + | + | + | + | P(a) | + | + | + | + | + | + | + | + |
| 21. DU 4475 | + | + | + | + | P(a) | + | + | + | + | + | + | + | + |
| 22. MB 361 | + | + | + | + | P(a) | + | + | + | + | + | + | + | + |
| 23. MB 415 | + | + | + | + | P(a) | + | + | + | + | + | + | + | + |
| 24. MB453 | + | + | + | + | + | + | + | + | + | + | + | + | + |
| 25. UACC 893 | + | + | + | + | + | + | + | + | + | + | + | + | + |
| 26. LNCAP | + | + | + | + | + | + | + | + | + | + | + | + | + |
| 27. PC-3 | + | + | + | + | + | + | + | + | + | + | + | + | + |
| Note: | | | | | | | | | | | | | |
| +: Identical to the wild-type MTG16 sequence. Exon 2: P(z) = c763C→A in MTG16a or c49C→A in MTG16b (P255T in MTG16a or P17T in MTG16b); Exon 4: P(a) = c954A→G in MTG16a or c165A→G in MTG16b (Ala→Ala; present in tumour and normal samples); | | | | | | | | | | | | | |
| # Exon 5: Intronic G→C polymorphism (+ = G, P(a) = G/C, P(b) = C); Exon10: P(a) = Intronic G→A change (RT-PCR results indicate no effect on splicing). | | | | | | | | | | | | | |

### REFERENCES

References cited herein are listed on the following pages, and are incorporated herein by this reference.
Alland, L. et al. (1997). Nature 387: 49-55.
Altschul, S F. et al. (1997). Nucleic Acids Res. 25: 3389-3402.
Brenner, A J. and Aldaz C M. (1995). Cancer Res. 55: 2892-2895.
Callen, D F. et al. (1990). Ann. Genet. 33: 219-221.
Callen, D F. et al. (1995). Genomics 29: 503-511.
Callen, D F. Et al. (2001). Cancer Genet. Cytogenet. (In Press).
Cavenee, W K. et al. (1983). Nature 305: 779-784.
Chen, T. et al. (1996). Cancer Res. 56: 5605-5609.
Cleton-Jansen, A-M. et al (1995). Br. J. Cancer 72: 1241-1244.
Cole, S P. et al. (1984). Mol. Cell Biol. 62: 109-120.
Cote, R J. et al. (1983). Proc. Natl. Acad. Sci. USA 80: 2026-2030.
Culver, K. (1996). Gene Therapy: A Primer for Physicians. Second Edition. (Mary Ann Liebert).
Devilee, P. et al. (1991). Oncogene 6: 1705-1711.
Devilee, P. et al. (1994). Genes Chrom. Cancer 11: 71-78.
Devilee, P. and Cornelisse, C J. (1994). Biochimica et Biophysica Acta 1198: 113-130.
Doggett, N A. et al. (1995). Nature 377 Suppl: 335-365.
Elston, C W. and Ellis, I O. (1990). Histopathology 16: 109-118.
Esteller, M. et al. (2000). J. Natl. Cancer Inst. 92: 564-569.
Fearon, E R. and Vogelstein, B. (1990). Cell 61: 759-767.
Friedman, T. (1991). In Therapy for Genetic Diseases. (T Friedman (Ed) Oxford University Press. pp 105-121.
Futreal, P A. et al. (1994). Science 266: 120-122.
Gamou, T. et al. (1998). Blood 91: 4028-4037.
Groden, J. et al. (1991). Cell 66: 589-600.
Hall, J M. et al. (1990). Science 250: 1684-1689.
Harlow, E. and Lane, D. (1988). Antibodies: A Laboratory Manual (Cold Spring Harbor Laboratory, Cold Spring Harbor, N.Y.).
Heinzel, T. et al. (1997). Nature 387: 43-48.
Heller, R A. et al. (1997). Proc. Natl. Acad. Sci. USA 94: 2150-2155.
Herman, J G. et al. (1998). Proc. Natl. Acad. Sci. USA 95: 6870-6875.
Huse, W D. et al. (1989). Science 246: 1275-1281.
Knudson, A G. (1971). Proc. Natl. Acad. Sci. USA 68: 820-823.
Kohler, G. and Milstein, C. (1975). Nature 256: 495-497.
Kozbor, D. et al. (1985). J. Immunol. Methods 81:31-42.
Longmire, J L. et al. (1993). GATA 10: 69-76.
McCormick, M K. et al. (1993). Proc. Natl. Acad. Sci. USA 90: 1063-1067.
Melnick, A M. et al. (2000). Mol. Cell Biol. 20: 2075-2086.
Miki, Y. et al. (1994). Science 266: 66-71.
Miki, Y. et al. (1996). Nature Genet. 13: 245-247.
Miyoshi, H. et al. (1991). Proc Natl Acad Sci USA 88:10431-10434.
Ohtani-Fujita, N. et al. (1997). Cancer Genet. Cytogenet. 98:43-49.
Orlandi, R. et al. (1989). Proc. Natl. Acad. Sci. USA 86: 3833-3837.
Prowse, A H. et al. (1997). Am. J. Hum. Genet. 60:765-771.
Radford, D M. et al. (1995). Cancer Res. 55: 3399-3405.
Riethman, H C. et al. (1989). Proc. Natl. Acad. Sci. USA 86: 6240-6244.
Saito, H. et al. (1993). Cancer Res. 53: 3382-3385.
Sambrook, J. et al. (1989). Molecular cloning: a laboratory manual. Second Edition. (Cold Spring Harbour Laboratory Press, New York).
Scharf, D. et al. (1994). Results Probl. Cell Differ. 20: 125-162.
Schena, M. et al. (1996). Proc. Natl. Acad. Sci. USA 93: 10614-10619.
Soares, M B. et al. (1994). Proc. Natl. Acad. Sci. USA 91: 9228-9232.
Sparkes, R S. et al. (1983). Science 219: 971-973.
Strassburg, C P. et al. (1997). Mol. Pharmacol. 52: 212-220.
Thiel, G. et al. (2000). Biochim. Biophis. Acta. 1493: 289-301.
Thompson, E W. et al. (1992). J. Cell Physiol. 150: 534-544.
Wang, J. et al. (1998). Proc Natl Acad Sci USA 95: 10860-10865.
Weber, J L. and May, P E. (1989). Am. J. Hum. Genet. 44: 388-396.
Whitmore, S A. et al. (1994). Genomics 20: 169-175.
Whitmore, S A. et al. (1998). Genomics 50: 1-8.
WHO. (1981). Histological Typing of Breast Tumours. Second Edition. (Geneva).
Winter, G. et al. (1991). Nature 349: 293-299.
Wooster, R. et al. (1994). Science 265: 2088-2090.
Wooster, R. et al. (1995). Nature 378: 789-791.
Wyman, A. and White, R. (1980). Proc. Natl. Acad. Sci. USA 77: 6754-6758.

1-101. (canceled)

102. An isolated DNA molecule comprising the nucleotide sequence set forth in SEQ ID NO:1.

103. An isolated DNA molecule, consisting of the nucleotide sequence set forth in SEQ ID NO:1, or a fragment thereof which encodes a polypeptide active in suppressing cellular functions associated with cancer, provided that said fragment includes some or all of nucleotides 1 to 453.

104. An isolated DNA molecule with at least 75% sequence identity to a DNA molecule consisting of the nucleotide sequence set forth in SEQ ID NO:1, or a fragment thereof, which includes some or all of nucleotides 1 to 453 and which encodes a polypeptide active in suppressing cellular functions associated with cancer.

105. An isolated DNA molecule as claimed in claim 104 with at least 85% sequence identity.

106. An isolated DNA molecule as claimed in claim 105 with at least 95% sequence identity.

107. An isolated DNA molecule that encodes a polypeptide active in suppressing cellular functions associated with cancer, and which hybridizes under stringent conditions with a DNA molecule consisting of the nucleotide sequence set forth in SEQ ID NO:1, at least in Dart through base pairing with some or all of nucleotides 1 to 453.

108. An isolated DNA molecule which encodes a polypeptide having the amino acid sequence set forth in SEQ ID NO:3.

109. An isolated DNA molecule which encodes a polypeptide active in suppressing cellular functions associated with cancer, the polypeptide having an amino acid sequence with at least 75% identity to that set forth in SEQ ID NO:3, or a fragment thereof, and which includes some or all of nucleotides 1 to 453 set forth in SEQ ID NO:1.

110. An isolated DNA molecule as claimed in claim 109 wherein the amino acid sequence has at least 85% sequence identity.

111. An isolated DNA molecule as claimed in claim 110 wherein the amino acid sequence has at least 95% sequence identity.

112. An isolated DNA molecule consisting of the nucleotide sequence set forth in SEQ ID NO:1

113. An isolated DNA molecule consisting of the nucleotide sequence set forth in SEQ ID NO:2.

114. An expression vector which comprises at least the coding sequence of a DNA molecule as defined in claim 102 or a DNA molecule as defined in claim 114 operably linked to suitable control elements.

115. Host cells transformed with the expression vector of claim 18.

116. An isolated DNA molecule encoding MTG16 which is inactivated or whose expression is down-regulated through epigenetic mechanisms or as a result of mutation or polymorphism.

117. An isolated DNA molecule as claimed in claim 117 which is abnormally methylated in the promoter region.

118. An isolated DNA molecule which encodes MTG16b and is abnormally methylated in the CpG island spanning exon 1b and the 5′UTR.

119. An isolated DNA molecule as claimed in claim 117 which encodes MTG16 including one or more polymorphisms selected from those disclosed in Tables 3-5.

120. A method for the diagnosis of cancer, or a predisposition to cancer, in a patient, comprising the steps of:
(a) establishing a profile for normal expression of MTG16 in unaffected subjects;
(b) measuring the level of expression of MTG16 in the patient: and
(c) comparing the measured level of expression of MTG16 in the patient with the profile for normal expression.

121. A method as claimed in claim 121 wherein reverse transcriptase PCR is employed to measure levels of expression.

122. A method as claimed in claim 121 wherein a hybridisation assay using a probe derived from MTG16, or a fragment thereof, is employed to measure levels of expression.

123. A method for the diagnosis of cancer, or a predisposition to cancer associated with mutations in MTG16 in a patient, comprising the steps of:
(a) obtaining a sample which includes MTG16 or a nucleic acid which codes for MTG16 from the patient:
(b) comparing MTG16 or a nucleic acid which codes for MTG16 from the sample with wild-type MTG16 or a nucleic acid which codes for it in order to establish whether the person expresses a mutant MTG16.

124. A method as claimed in claim 124 wherein the nucleotide sequence of DNA from the patient is compared to the sequence of DNA encoding wild-type MTG16.

125. A method as claimed in claim 124 wherein the electrophoretic mobility of MTG16 from the patient and wild-type MTG16 is compared.

126. A genetically modified non-human animal in which MTG16 activity is reduced or absent.

127. A genetically modified non-human animal as claimed in claim 127 in which MTG16 gene function has been knocked out.

128. A genetically modified non-human animal in which MTG16 gene function is modified through transformation with a mutant MTG16 gene.

129. A genetically modified non-human animal as claimed in claim 127 in which the animal is selected from the group consisting of rats, mice, hamsters, guinea pigs, rabbits, dogs, cats, goats, sheep, pigs and non-human primates such as monkeys and chimpanzees.

130. A genetically modified non-human animal as claimed in claim 126 wherein the animal is a mouse.
